(19) United States
(12) Patent Application Publication
(10) Pub. No.: US 2022/0308070 A1
(43) Pub. Date: Sep. 29, 2022

(54) MONITORING GENE THERAPY

(71) Applicant: LogicBio Therapeutics, Inc., Lexington, MA (US)

(72) Inventors: B. Nelson Chau, Needham, MA (US); Jing Liao, Lexington, MA (US); Susana Gordo, Boston, MA (US)

(21) Appl. No.: 17/603,935

(22) PCT Filed: Apr. 14, 2020
(86) PCT No.: PCT/US20/28102

§ 371 Date: Oct. 14, 2021

Related U.S. Application Data
(60) Provisional application No. 62/833,875, filed on Apr. 15, 2019.

(51) Int. Cl. G01N 33/68; C12N 15/90

(52) U.S. Cl. CPC G01N 33/6893; C12N 15/907; C12N 2750/14143; G01N 2800/52

(57) ABSTRACT

The present disclosure provides, among other things, technologies for improving gene therapy. Among other things, the present disclosure provides technologies that permit monitoring and/or assessment one or more characteristics of a gene therapy treatment such as, for example, extent, level, and/or persistence of payload expression. In some embodiments, provided technologies particularly useful with integrating gene therapy.

### CROSS REFERENCE TO RELATED APPLICATIONS

This application claims priority to U.S. Provisional Application No. 62/833,875 filed Apr. 15, 2019, the content of which is herein incorporated by reference in its entirety.

### SEQUENCE LISTING

The instant application contains a Sequence Listing which has been submitted electronically as a .txt file named “2012538-0082_SL.txt”. The .txt file was created on Apr. 10, 2020 and is 26,104 bytes in size. The entire contents of the Sequence Listing are herein incorporated by reference.

### BACKGROUND

There is a subset of human diseases that can be traced to changes in the DNA that are either inherited or acquired early in embryonic development. Of particular interest for developers of genetic therapies (also referred to as “gene therapies”) are diseases caused by a mutation in a single gene, known as monogenic diseases. There are believed to be over 6,000 monogenic diseases. Typically, any particular genetic disease caused by inherited mutations is relatively rare, but taken together, the toll of genetic-related disease is high. Well-known genetic diseases include cystic fibrosis, Duchenne muscular dystrophy, Huntington's disease and sickle cell anemia. Other classes of genetic diseases include metabolic disorders, such as organic acidemias, and lysosomal storage diseases where dysfunctional genes result in defects in metabolic processes and the accumulation of toxic byproducts that can lead to serious morbidity and mortality both in the short-term and long-term.

While gene therapy has received a lot of attention in terms of development of therapeutic candidates, much less attention has been directed to ways of monitoring and assessing the effectiveness and trajectory of gene therapies. What is needed are new methods and compositions for ensuring gene therapy is applied effectively, including in conjunction with one or more additional treatments.

### SUMMARY

The present disclosure provides, among other things, technologies for improving gene therapy. Among other things, the present disclosure provides technologies that permit monitoring and/or assessment of one or more characteristics of a gene therapy treatment such as, for example, extent, level, and/or persistence of payload expression. In some embodiments, provided technologies particularly useful with integrating gene therapy.

Among other things, the present disclosure demonstrates that certain integrating gene therapy technologies can generate novel biomarker entities whose expression and/or activity may be highly correlated with expression and/or activity of a payload (e.g., of a product encoded by and/or expressed from a transgene) of interest delivered by the gene therapy. Moreover, the present disclosure teaches that such generation can provide strategies for monitoring and/or otherwise assessing the gene therapy and/or its success, stability, maintenance, etc.

Among other things, it has been found, in some embodiments, that presently disclosed biomarkers can be assessed directly from a biological sample taken non-invasively from a subject that has received the gene therapy and that assessment of such biomarkers can provide information about the status of a payload that would otherwise require more invasive procedures to determine. As but one example, the present disclosure demonstrates that one need not perform a tissue biopsy in order to determine delivery or expression of a payload in the tissue. Rather, analysis of a biomarkers from the circulation (e.g., via a blood draw) can be evaluated to indirectly reveal one or more aspects of the expression and/or activity of the payload in the tissue. This can simplify and facilitate analysis of payloads delivered to intracellular locations.

In some embodiments, the present disclosure provides methods of monitoring gene therapy, the methods including a step of detecting, in a biological sample from a subject who has received integrating gene therapy treatment, a level or activity of a biomarker generated by integration of the integrating gene therapy treatment, as a surrogate for one or more characteristics of the status of the gene therapy treatment, wherein the one or more characteristics of the status of the gene therapy treatment is selected from the group consisting of level of a payload, activity of a payload, level of integration of the gene therapy treatment in a population of cells, and combinations thereof.

In some embodiments, the present disclosure provides methods of monitoring delivery, level and/or activity of a payload in a subject who has received a gene-integrating composition that delivers the payload, the methods including a step of detecting, in a biological sample from the subject, level or activity of a biomarker generated by integration of the gene-integrating composition, as a surrogate for delivery, level and/or activity of the payload.

In some embodiments, the present disclosure provides methods of determining one or more characteristics of the status of gene therapy treatment in a subject who has received an integrating gene therapy treatment, the methods including the steps of a) providing a biological sample from the subject, b) determining a level of a biomarker, wherein the biomarker is generated by integration of the gene therapy in the genome of the subject, and c) based on the determined level of the biomarker, establishing one or more characteristics of the status of gene therapy treatment in the subject, wherein the determined level of the biomarker corresponds one or more characteristics of the status of gene therapy treatment.

In some embodiments, the present disclosure provides methods of delivering a gene therapy treatment to a subject in need thereof, including the steps of a) administering an integrating gene therapy treatment to the subject, and b) determining in a biological sample from the subject a level of a biomarker that is generated by integration of the gene therapy treatment in the genome of the subject.

In some embodiments, an integrating gene therapy treatment or gene-integrating composition achieves integration of a nucleic acid element comprising a sequence that encodes a payload into a target site in the genome of the subject. Those of skill in the art will appreciate that any of a variety of target sites may be appropriate for use with methods and compositions as described herein. For example, in some embodiments, a target site encodes a polypeptide (e.g., albumin). In some embodiments, integration of the nucleic acid element occurs at the 5′ or 3′ end of a gene that encodes a polypeptide. In some embodiments, a target site encodes albumin.

In accordance with various embodiments, any application-appropriate payload may be used as described herein. In some embodiments, a payload is or comprises a peptide/polypeptide/protein, a nucleic acid (e.g., shRNA, miRNA), and any combination thereof. For example, in some embodiments, a payload is or comprises a peptide expressed intracellularly. In some embodiments, a payload is or comprises a peptide that is secreted extracellularly. In some embodiments, a payload is a peptide that has cell-intrinsic or cell-extrinsic activity that promotes a biological process to treat a medical condition. In some embodiments, a payload is a peptide that is normally expressed in liver cells. In some embodiments, a payload is a peptide that is ectopically expressed in liver cells. In some embodiments, a payload is methylmalonyl-CoA mutase, alpha-1-antitrypsin, or human Factor IX.

As is described herein, many embodiments include the use of one or more biological samples (e.g., a sample of fluid or tissue taken from a subject). In accordance with the present disclosure, any of a variety of biological samples are contemplated as compatible with various embodiments. For example, in some embodiments, a biological sample is or comprises hair, skin, feces, blood, plasma, serum, cerebrospinal fluid, urine, saliva, tears, vitreous humor, or mucus.

As is described herein, detecting (e.g., detecting a signal, such as a biomarker or detectable moiety), as applicable to methods and compositions described herein, may be achieved in any application-appropriate manner. For example, in some embodiments, a step of detecting is or comprises an immunological assay or a nucleic acid amplification assay.

As is described in the present disclosure, use of a variety of biomarkers is contemplated as compatible with various embodiments. In some embodiments, a biomarker is or comprises a detectable moiety that, after translation of a polypeptide encoded by a target site, becomes fused to the polypeptide encoded by the target site. In some embodiments, a biomarker is or comprises a detectable moiety that, after translation of a polypeptide encoded by a target site, becomes fused to the polypeptide encoded by a payload. In some embodiments, a biomarker is or comprises a 2A peptide. In some embodiments, a 2A peptide is selected from the group consisting of P2A, T2A, E2A and F2A. In some embodiments, a biomarker may be or comprise a Furin cleavage motif. In some embodiments, a detectable moiety may be or comprise an agent that binds to a biomarker (e.g., an antibody or fragment thereof).

In some embodiments, integration of a nucleic acid element does not significantly disrupt expression of the polypeptide encoded at the target site (i.e., expression of the polypeptide at the target site continues substantially as it would have had the subject not received the integrating gene therapy treatment or gene-integrating composition). In some embodiments, integration occurs without the use of an exogenously supplied nuclease. In some embodiments, integration occurs with the use of one or more exogenously supplied nucleases.

In accordance with various embodiments, methods and compositions described herein are contemplated as compatible with a variety of gene therapy regimen. For example, in some embodiments, a subject receives a single dose of a gene therapy treatment or gene-integrating composition. In some embodiments, a subject receives multiple doses of a gene therapy treatment or gene-integrating composition (e.g., 2, 3, 4, 5, 6, 7, 8, 9, 10 or more).

Additionally, methods and compositions as described herein are contemplated as applicable at any of a variety of times post gene therapy treatment (e.g., hours, days, weeks, or months after the subject receives a gene therapy). Accordingly, in some embodiments, a detecting step is performed 1, 2, 3, 4, 5, 6, 7, 8 or more weeks after the subject has received the gene therapy treatment or gene-integrating composition. In some embodiments, a detecting step is performed at multiple time points after the subject has received the gene therapy treatment or gene-integrating composition. In some embodiments, a detecting step is performed (e.g., multiple times) over a period of at least 3 months after the subject has received the gene therapy treatment or gene-integrating composition.

Surprisingly, it was found that some embodiments are capable of providing benefit (e.g., facilitating monitoring and/or adjustment of therapy) to a subject that is at various stages of life when receiving a gene therapy and, in some embodiments, provided methods may be used as a subject transitions between stages of life. In some embodiments, a subject receives the gene therapy treatment or gene-integrating composition as an infant. In some embodiments, a subject receives the gene therapy treatment or gene-integrating composition before reaching adulthood (e.g., as a child). In some embodiments, a subject receives the gene therapy treatment or gene-integrating composition as an adult.

It is specifically contemplated that methods and compositions as described herein are applicable to a variety of subjects, each potentially having confounding or complicating factors/conditions in addition to those necessitating the application of gene therapy. In addition, some forms of gene therapy are known or suspected of potentially causing problematic reactions (e.g., autoimmune reactions, cytokine storms, etc). Accordingly, in some embodiments, provided methods further comprise monitoring the subject for autoimmune response to the gene therapy. In some embodiments, provided methods further comprise monitoring the subject for an abnormal cytokine response to the gene therapy (e.g., a cytokine storm).

The present disclosure also encompasses the recognition that gene therapy may need to be adjusted at times (e.g., enhanced or suppressed), and it is contemplated that various embodiments are advantageous in monitoring the need for, and/or successfully making such adjustments. Accordingly, in some embodiments, provided methods further comprise administering an additional treatment (e.g., an activating agent) to the subject if the level of the biomarker is lower than would indicate a therapeutically effective amount of the integrating gene therapy has been achieved. Additionally or alternatively, in some embodiments, provided methods further comprise delivering an additional treatment (e.g., a deactivating agent) to the subject that reduces or inhibits expression of a payload delivered by the gene therapy treatment if the level of the biomarker exceeds a level that is indicative of an optimal or safe level of the payload.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 shows a schematic of the GeneRide™ construct (AAV) before integration and following HR-mediated integration into the genome at the targeted albumin, Alb, locus. Expression from the GeneRide™-edited Alb locus can result in the simultaneous production of albumin-2A and the transgene as separate proteins.

FIG. 2 depicts exemplary methods for analysis of genomic DNA (gDNA) integration. As illustrated, such methods can be applied to assay for GeneRide™-edited gDNA in the albumin (Alb) locus. In depicted Step 1, long-range PCR (LR-PCR) amplifies product from isolated gDNA with primers F1/R1. In depicted Step 2, purified product from Step 1 is amplified with primers F2/R2 in a nested qPCR.

FIG. 3 presents an exemplary approach for quantification of episomal DNA. As illustrated, episomal copy numbers can be determined by qPCR using a standard curve built with linearized episomal plasmid.

FIG. 4 presents an exemplary approach for analysis of mRNA comprising a nucleic acid sequence encoding 2A peptide. Fused mRNA copy number is determined by ddPCR with primer set Fwd/R_{F}. Endogenous Alb copy number is measured by ddPCR with primer set Fwd/R_{E} and used for normalization.

FIG. 5A-FIG. 5B present an exemplary approach for detection and quantification of polypeptides in plasma. For example, Albumin-2A in plasma can be analyzed via the illustrated methods. FIG. 5A) depicts a sandwich ELISA comprising a capture antibody and a detection antibody. In the illustration, an anti-2A antibody for capture of albumin-2A and a labeled anti-albumin antibody for detection are presented. Capture and detection antibodies specific for other polypeptides, such as albumin; human Factor IX; and cyno A1AT can be used to detect other such polypeptides in plasma. FIG. 5B) depicts standard curves based on recombinant mouse albumin-2A in PBST buffer or 10% mouse serum.

FIG. 6A-FIG. 6C demonstrate detection and analysis of episomal DNA in vivo. Neonatal mice (p2) were injected with 1e14 vg/kg of hF9-DJ. FIG. 6A) Episomal copy numbers decrease exponentially over time after injection. FIG. 6B) Liver growth of animals is not significantly affected after injection. FIG. 6C) Growth in body weight of the animals is not significantly affected after injection.

FIG. 7A-FIG. 7D demonstrate in vivo detection, monitoring and analysis of a 2A biomarker over time. Neonate C57 mice were injected i.v. at p2 with 1e14 vg/kg of hF9-DJ, and harvested at 1, 2, 3, 4 and 8 weeks post-injection (n=5/group). FIG. 7A) Genomic DNA integration of 2A biomarker in liver was quantified by LR-PCR/qPCR and expressed as a percent of endogenous Alb. FIG. 7B-FIG. 7C) ALB-2A and total mouse albumin in plasma were measured by ELISA. FIG. 7D) Correlation of data presented in FIG. 7B and FIG. 7C. Analysis of 2A peptide-tagged albumin versus total albumin in plasma confirms that the observed increase in plasma ALB-2A is associated to the exponential increase of endogenous albumin after birth.

FIG. 8A-FIG. 8B demonstrate in vivo detection, monitoring and analysis of a payload delivered with a biomarker over time. Neonate C57 mice were injected i.v. at p2 with 1e14 vg/kg of hF9-DJ, and harvested at 1, 2, 3, 4 and 8 weeks post-injection. FIG. 8A) Human Factor IX was quantified in mouse plasma by a human-specific Factor IX ELISA. FIG. 8B) Correlation of data presented in FIG. 7B and FIG. 8A.

FIG. 9A-9E demonstrate detection and analysis of biomarker and payload delivery at two doses and two age groups. Neonate C57 mice (p2) or juvenile mice (p21) were injected i.v. with 1e13 or 1e14 vg/kg of hF9-DJ, and harvested 8 weeks post-injection (n=6-8/group). At harvest, the age of the animals dosed at p2 was 8 weeks and those dosed at p21 were 11-week-old. FIG. 9A) ALB-2A in plasma measured by ELISA. FIG. 9B) Human Factor IX in plasma measured by ELISA. FIG. 9C) Fused mRNA in liver quantified by ddPCR and expressed as a percent of endogenous albumin mRNA. FIG. 9D) Genomic DNA integration in liver quantified by LR-PCR/qPCR and expressed as a percent of endogenous Alb. FIG. 9E) Episomal copy numbers per cell measured in liver by qPCR.

FIG. 10A-FIG. 10C demonstrate in vivo detection, monitoring and analysis of a payload delivered with a biomarker to subjects in different age groups. Neonate FvB/NJ (p2), juvenile (p21), or adult mice (p42 and p63) were dosed i.v. with 1e14 vg/kg of hF9-DJ and harvested 4 weeks post-injection (n=6-9/group). At harvest, the age of the animals dosed at p2 was 4 weeks, those dosed at p21 were 7-weeks-old, those dosed at p42 were 11-weeks-old, and those dosed at p63 were 14-week-old. FIG. 10A-FIG. 10B) ALB-2A and human Factor IX in plasma were measured by ELISA. FIG. 10C) Linear regression of plasma ALB-2A vs human Factor IX yields a R²=0.93.

FIG. 11A-FIG. 11C demonstrate in vivo detection, monitoring and analysis of a payload delivered with a biomarker via viral vectors comprising different homology arms for genomic integration. Neonate FvB/NJ mice (p2) were injected i.v. with A1AT-DJ for a final dose of 1e13 or 1e14 vg/kg. HA-750 bp corresponds to a transgene with 750 bp homology arms and HA-1 kb corresponds to a transgene with 1 kb homology arms. Animals were harvested 6 weeks post-injection (n=6-9/group). FIG. 11A-FIG. 11B) ALB-2A and cyno A1AT in mouse plasma were measured by ELISA. FIG. 11C) Linear regression of ALB-2A vs A1AT yields a R²=0.91.

FIG. 12A-FIG. 12B demonstrate in vivo detection, monitoring and analysis of a cell-intrinsic payload delivered with a biomarker. Neonatal Mut^{-/-}; Tg^{INS-MCK-Mut} mice (p2) were injected i.v. with different doses of DJ-hMUT (1e13, 3e13 or 1e14 vg/kg) and harvested over a period of 3 months. FIG. 12A) Genomic DNA integration in liver was quantified by LR-PCR/qPCR and is expressed as a percent of endogenous Alb. ALB-2A in plasma was measured by ELISA. FIG. 12B) Protein expression of the integrated transgene, human MUT, was measured by Western blot in liver lysates of MCK-MUT mice. Circulating ALB-2A appears to linearly correlate with the levels of genomic integration in liver as well as the levels of MUT protein.

FIG. 13A-FIG. 13B demonstrate that expression of a payload can increase after a single administration (i.e. selective expansion of GeneRide™-edited hepatocytes) and that an increase in payload levels can be monitored by analysis of biomarker levels. Neonatal Mut^{-/-};Tg^{INS-MCK-Mut} mice (p2) were injected i.v. with 1e14 vg/kg of DJ-hMUT, and harvested mice over a period of 7 months. FIG. 13A-13B) Human MUT protein expressed from the integrated transgene in MUT^{-/-} mice was analyzed in liver lysates by Western blot, using β-actin as a loading control. ALB-2A (blotting for 2A) and total albumin were also analyzed in these liver lysates. Vehicle-treated MUT^{+/-} and wild-type B6 mice were analyzed as reference. Note: MUT protein in MUT^{-/-} hepatocytes expressed from the transgene is human while endogenous protein in MUT^{+/-} and wild-type B6 is mouse.

FIG. 14 demonstrates standard curves based on recombinant mouse ALB-2A prepared in sample diluent only or 1% mouse plasma, utilizing an optimized ALB-2A ELISA method.

FIG. 15A-15B demonstrate in vivo detection, monitoring and analysis of a payload delivered with a biomarker in wild-type mice and a mouse model of NAFLD (DIO).

Adult mice (˜9-week-old) were injected i.v. with 1e14 vg/kg of hF9-DJ, and plasma samples were collected at week 1 and biweekly thereafter for a total of 16 weeks. ALB-2A and human Factor IX were quantified in mouse plasma by ELISA.

FIG. 16A-16C demonstrate detection and analysis of biomarker and payload delivery in a dose-dependent manner. Neonate FvB mice (p2) were injected i.v. with 4.1e12, 1.2e13, 3.7e13, 1.1e14, 3.3e14, and 1e15 vg/kg of cA1AT-DJ, and harvested 4 weeks post-injection (n=5/group). FIG. 16A-B) show ALB-2A and cA1AT levels in plasma measured by ELISA, respectively. FIG. 16C) shows a linear regression of ALB-2A vs A1AT yields R²=0.97.

FIG. 17A-C demonstrate detection and analysis of gDNA integration and payload delivery in a dose-dependent manner. Neonatal Mut^{+/-}; Tg^{INS-MCK-Mut} mice (p0) were injected i.v. with a low, mid and high dose of DJ-mMUT (2.1e13, 6.7e13 or 2.0e14 vg/kg) and harvested 90 days post-dosing. FIG. 17A shows ALB-2A levels in plasma measured by ELISA (n=18 low dose, n=19 mid dose, n=19 high dose). FIG. 17B shows percent gDNA integration in liver (n=26 low dose, n=25 mid dose, n=28 high dose). FIG. 17C shows correlation of ALB-2A and percent gDNA integration for animals with both analyses.

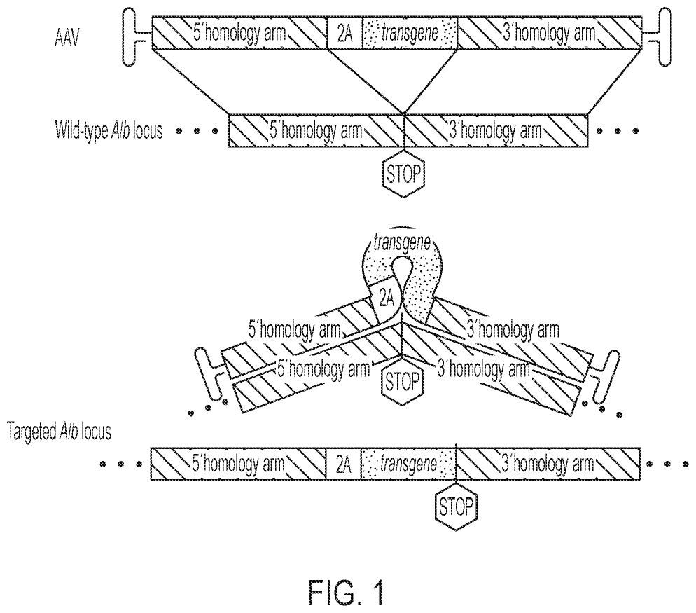

### DEFINITIONS

In order for the present invention to be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control). In some embodiments, an activating agent binds or otherwise associates with an activating element in order to exert its effect.

Adult: As used herein, the term “adult” refers to a human eighteen years of age or older. In some embodiments, a human adult has a weight within the range of about 90 pounds to about 250 pounds.

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to an entity whose presence, level, or form correlates with a particular biological event or state of interest, so that it is considered to be a “marker” of that event or state. Among other things, the present disclosure provides biomarkers for gene therapy (e.g., that are useful to assess one or more features or characteristics of a gene therapy treatment, such as, for instance, extent, level, and/or persistence of payload expression). In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc). In certain embodiments, the present disclosure demonstrates effectiveness of biomarkers that can be detected in a sample obtained from a subject who has received gene therapy for use in assessing one or more features or characteristics of that gene therapy; in some such embodiments, the sample is of cells, tissue, and/or fluid other than that to which the gene therapy was delivered and/or other than that where the payload is active.

Detectable Moiety: The term “detectable moiety” as used herein refers to any entity (e.g., molecule, complex, or portion or component thereof). In some embodiments, a detectable moiety is provided and/or utilizes as a discrete molecular entity; in some embodiments, it is part of and/or associated with another molecular entity. Examples of detectable moieties include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, antibodies, and/or proteins for which antisera or monoclonal antibodies are available.

Child: As used herein, the term “child” refers to a human between two and 18 years of age. Body weight can vary widely across ages and specific children, with a typical range being 30 pounds to 150 pounds.

Combination therapy: As used herein, the term “combination therapy” refers to those situations in which a subject is simultaneously exposed to two or more therapeutic regimens (e.g., two or more therapeutic agents, for example a gene therapy and a non-gene therapy therapeutic modality). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time).

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Deactivating agent: As used herein, the term “deactivating agent” refers to an agent whose presence or level correlates with a decreased level or activity of a target, as compared with that observed absent the agent (or with the agent at a different level). In some embodiments, a deactivating agent is one whose presence or level correlates with a target level or activity that is comparable to or lower than a particular reference level or activity (e.g., that observed under appropriate reference conditions, such as presence of a known activating agent, e.g., a positive control). In some embodiments, a deactivating agent binds or otherwise associates with an deactivating element in order to exert its effect.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Gene: As used herein, the term “gene” refers to a DNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Infant: As used herein, the term “infant” refers to a human under two years of age. Typical body weights for an infant range from 3 pounds up to 20 pounds.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Peptide: As used herein, the term “peptide” or “polypeptide” refers to any polymeric chain of amino acids. In some embodiments, a peptide has an amino acid sequence that occurs in nature. In some embodiments, a peptide has an amino acid sequence that does not occur in nature. In some embodiments, a peptide has an amino acid sequence that is engineered in that it is designed and/or produced through action of the hand of man. In some embodiments, a peptide may comprise or consist of natural amino acids, non-natural amino acids, or both. In some embodiments, a peptide may comprise or consist of only natural amino acids or only non-natural amino acids. In some embodiments, a peptide may comprise D-amino acids, L-amino acids, or both. In some embodiments, a peptide may comprise only D-amino acids. In some embodiments, a peptide may comprise only L-amino acids. In some embodiments, a peptide is linear. In some embodiments, the term “peptide” may be appended to a name of a reference peptide, activity, or structure; in such instances it is used herein to refer to peptides that share the relevant activity or structure and thus can be considered to be members of the same class or family of peptides. For each such class, the present specification provides and/or those skilled in the art will be aware of exemplary peptides within the class whose amino acid sequences and/or functions are known; in some embodiments, such exemplary peptides are reference peptides for the peptide class or family. In some embodiments, a member of a peptide class or family shows significant sequence homology or identity with, shares a common sequence motif (e.g., a characteristic sequence element) with, and/or shares a common activity (in some embodiments at a comparable level or within a designated range) with a reference peptide of the class; in some embodiments with all peptides within the class). For example, in some embodiments, a member peptide shows an overall degree of sequence homology or identity with a reference peptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Polypeptide: As used herein, the term “polypeptide” or “protein” refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION OF CERTAIN EMBODIMENTS

The present disclosure provides technologies for monitoring and/or otherwise assessing gene therapy. As described herein, the present disclosure relates to detection and assessment of biomarker(s) that are generated as a result of integrating gene therapy treatment, the presence and relative amounts of which reveal information about a payload delivered via the gene therapy treatment, e.g., information about the presence, amount, and/or kinetics of the delivered payload. In one aspect of the present disclosure, the presence, amount, and/or kinetics of a biomarker acts as a proxy for the determination of presence, amount, and/or kinetics of the delivered payload. In some embodiments of the disclosure, a biomarker is assessed from a biological sample taken from a subject who has received an integrating gene therapy treatment.

In some embodiments of the disclosure, a biomarker can be assessed in a non-tissue biological sample taken from the subject. In some embodiments of the disclosure, a payload is delivered to (e.g., through delivery of an appropriate transgene) and remains within a tissue of a subject who has received an integrating gene therapy treatment.

### Integrating Gene Therapy

Gene therapy introduces genetic material into cells of a subject, typically in order to express a payload that can compensate for an abnormal gene or to otherwise provide a beneficial effect to the subject. Integrating gene therapy introduces genetic material that becomes integrated into a genetic sequence (i.e., a target site) present in the recipient cell.

Those skilled in the art are aware of a variety of technologies for integrating genetic material into a target site of interest in a recipient cell or organism. Such integrated genetic elements can comprise a nucleic acid sequence (i.e., “transgene” as that term is used herein) that encodes a payload to be delivered to the host cell or organism. Typically, a transgene is delivered in the context of a vector; those skilled in the art are aware of both viral and non-viral vector systems that can successfully be employed to achieve transgene integration.

The present disclosure provides the identification of the source of a problem with various integrating gene therapy technologies.

For example, the present disclosure appreciates that inefficient or ineffective integration can limit usefulness of gene therapy strategies. If a vector fails to integrate, it will typically be lost when cells divide during the process of growth or tissue regeneration, and any benefits that are or would have been provided by the delivered transgene (or payload) will also be lost. A similar difficulty arises even when an integration is initially successful, but subsequently lost, for example via a recombination event or by death of a recipient cell.

The present disclosure further appreciates that many gene integration technologies or events cannot or not precisely control target integration site, and that site of integration can significantly impact degree and/or timing of transgene expression and/or can impact health or even viability of the receiving cell. Furthermore, the present disclosure appreciates that even some “targeted” gene integration technologies may be negatively impacted by site of integration, such that transgene expression may fail to achieve and/or be maintained at a desired level and/or for a desired period of time.

The present disclosure further appreciates that many gene therapy approaches introduce a transgene in operative association with a promoter (e.g., an exogenous promoter), and that expression characteristics of such a promoter can negatively impact recipient cells, including by potentially increasing the risk of uncontrolled proliferation (e.g., cancer), particularly for promoters that drive high levels of gene expression.

Thus, the present disclosure provides an insight that the source of one problem with many integrating gene therapy treatments is the failure or inability to monitor expression of the relevant payload, particularly over time. Given that many payloads are or may be intracellular and/or that tissues in which they are intended to be expressed and/or active may be relatively inaccessible, regular monitoring is often not attempted.

The present disclosure contributes a finding that certain gene integration technologies can generate an effective biomarker for successful transgene integration and payload expression. Moreover, the present disclosure demonstrates that certain such technologies generate a biomarker that can be assessed from readily accessible biological samples (e.g., blood, urine, tears, etc). Thus, the present disclosure provides technologies that improve integrating gene therapy, among other things by providing systems for monitoring (e.g., detecting and/or quantifying, in many embodiments at multiple points in time) a biomarker generated by successful integration and reflective of payload expression.

It is contemplated that any of a variety of integrative gene therapy technologies may be used. By way of non-limiting example, in some embodiments, an integrative gene therapy may be or comprise use of a vector-based systems (e.g., viral vector-based systems), a non-viral vector based system, a nuclease-mediated system, and/or use of a GENERIDE™ system, or any combination thereof.

### Vector-Based Systems

In some embodiments, an integrating gene therapy may be or comprise a vector-based system (e.g., a viral vector). Typically, a vector-based system will include a virus or viral genetic material into which a fragment of foreign DNA can be inserted for transfer into a cell. Any virus that includes a DNA stage in its life cycle may be used as a viral vector within the scope of some embodiments of the present disclosure. By way of non-limiting example, a viral vector-based system may be a single strand DNA virus, a double stranded DNA virus, an RNA virus that has a DNA stage in its life cycle, for example, retroviruses. In some embodiments, a viral vector may be delivered via a pharmaceutically acceptable formulation, for example, a liposome or lipid particle (e.g., a micro- or nano-particle).

As one non-limiting example, one virus of interest is adeno-associated virus. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

Regardless of the vector used (e.g., a viral vector), to promote targeted integration, the targeting vector comprises nucleic acid sequences that are permissive to homologous recombination at the site of integration, e.g. sequences that are permissive to homologous recombination with the albumin gene, a collagen gene, an actin gene, etc. This process requires nucleotide sequence homology, using the “donor” molecule, e.g. the targeting vector, to template repair of a “target” molecule i.e., the nucleic acid into which the nucleic acid of sequence is integrated, e.g. a target locus in the cellular genome, and leads to the transfer of genetic information from the donor to the target. As such, in targeting vectors of the subject compositions, the transgene to be integrated into the cellular genome may be flanked by sequences that contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the target integration site, to support homologous recombination between it and the genomic sequence to which it bears homology. Approximately 25, 50, 100, 250, or 500 nucleotides or more of sequence homology between a donor and a genomic sequence will support homologous recombination there between.

### Non-Viral Vector Systems

In some embodiments, an integrating gene therapy may be or comprise a non-viral-vector-based system. In some embodiments, a non-viral vector systems may be or comprise a plasmid, polymer-based particle, ceDNA, liposome, minicircle, and combinations thereof. In some embodiments, a non-viral vector system may be or comprise use of chemical carrier(s), electroporation, use of ballistic DNA (e.g., particle bombardment), sonoporation, photoporation, magnetofection, hydroporation, and any combination thereof.

Similar to the description above, regardless of the type(s) of non-viral vector system(s) used, to promote targeted integration, one or more nucleic acid sequences that are permissive to homologous recombination at the site of integration must be used/delivered to the target site.

### Nuclease-Mediated Integration

In accordance with various embodiments, nuclease-mediated integration uses one or more nucleases, enzymes that were engineered or initially identified in bacteria that cut DNA. Typically, nuclease- mediated integration is a two-step process. First, an exogenous nuclease, which is capable of cutting one or both strands in the double-stranded DNA, is directed to the desired site by a synthetic guide RNA and makes a specific cut. After the nuclease makes the desired cut or cuts, the cell's DNA repair machinery is activated and completes the editing process through either NHEJ or, less commonly, HDR.

In some embodiments, NHEJ can occur in the absence of a DNA template for the cell to copy as it repairs a DNA cut. This is the primary or default pathway that the cell uses to repair double-stranded breaks. The NHEJ mechanism can be used to introduce small insertions or deletions, known as indels, resulting in the knocking out of the function of the gene. NHEJ creates insertions and deletions in the DNA due to its mode of repair and can also result in the introduction of off-target, unwanted mutations including chromosomal aberrations.

Nuclease-mediated HDR occurs with the co-delivery of the nuclease, a guide RNA and a DNA template that is similar to the DNA that has been cut. Consequently, the cell can use this template to construct reparative DNA, resulting in the replacement of defective genetic sequences with correct ones. In some embodiments, an HDR mechanism is a preferred repair pathway when using a nuclease-based approach to insert a corrective sequence due to its high fidelity. However, a majority of the repair to the genome after being cut with a nuclease continues to use the NHEJ mechanism. The more frequent NHEJ repair pathway has the potential to cause unwanted mutations at the cut site, thus limiting the range of diseases that any nuclease- mediated integration approaches can target at this time.

### GeneRide™ Technology Platform

GeneRide™ is a genome editing technology that harnesses homologous recombination, or HR, a naturally occurring DNA repair process that maintains the fidelity of the genome. By using HR, GeneRide™ allows insertion of polynucleotides into specific targeted genomic locations without using exogenous nucleases, GeneRide™-directed polynucleotide integration is designed to leverage endogenous promoters at these targeted locations to drive high levels of tissue-specific gene expression, without the detrimental issues that have been associated with the use of exogenous promoters. In some embodiments of the present disclosure, In some embodiments of the present disclosure, GeneRide™ is used to deliver a polynucleotide that encodes a payload to a host cell or organism.

GeneRide™ technology can be used to precisely integrate a polynucleotide encoding a therapeutic payload into a patient's genome to provide a stable therapeutic effect. Because GeneRide™ is designed to have this durable therapeutic effect, it can be applied to targeting disorders in pediatric patients where it is critical to provide treatment early in a patient's life before irreversible disease pathology can occur.

In some embodiments, GeneRide™ uses an AAV vector to deliver a gene into the nucleus of a cell. It then uses HR to stably integrate the corrective gene into the genome of the recipient at a location where it is regulated by an endogenous promoter, leading to the potential for lifelong protein production, even as the body grows and changes over time.

GeneRide™ can provide precise, site-specific, stable and durable integration of a corrective gene into the chromosome of a host cell. In preclinical animal studies with GeneRide constructs, integration of the corrective gene in a specific location in the genome is observed.

The modular of GeneRide™ can be applied to deliver robust, tissue-specific gene expression that will be reproducible across different therapeutics delivered to one or more tissues. By substituting a different transgene within the GeneRide™ construct, that transgene can be delivered to address a new therapeutic indication while substantially maintaining all other components of the construct. This approach will allow leverage of common manufacturing processes and analytics across different GeneRide™ product candidates and could shorten the development process of other treatment programs.

Previous work on non-disruptive gene targeting is described in WO 2013/158309, incorporated herein by reference. Previous work on genome editing without nucleases is described in WO 2015/143177, incorporated herein by reference.

### Target Site

Integrating gene therapy for use in accordance with the present disclosure desirably achieves integration that achieves operative association of an integrated transgene with an active endogenous promoter, so that transcription from the promoter generates a transcript that extends through the transgene. Moreover, in many embodiments, integration is at a target site selected so that such a transcript includes an open reading frame other than that for the transgene.

In many embodiments, integrating gene therapy for use in accordance with the present disclosure achieves integration at a target site in an endogenous gene (e.g., at a specific position within or adjacent to an endogenous gene), and extends the transcript generated by transcription from that gene's promoter at least so that it extends through the transgene.

In some embodiments, an integrating gene therapy treatment or gene-integrating composition achieves integration of a nucleic acid element comprising a sequence that encodes a payload into a target site in the genome of the subject. Those of skill in the art will appreciate that any of a variety of target sites may be appropriate for use with methods and compositions as described herein. For example, in some embodiments, a target site encodes a polypeptide. In some embodiments, a target site may encode a polypeptide that is highly expressed in a subject (e.g., a subject not suffering from a disease, disorder or condition). In some embodiments, integration of the nucleic acid element occurs at the 5′ or 3′ end of an endogenous gene that encodes a polypeptide. By way of non-limiting example, in some embodiments, a target site encodes albumin.

It is contemplated that integrative delivery of genetic elements and/or transgenes can be accomplished for any tissue, including, but not limited to the liver, central nervous system (e.g., spine), muscle, kidney, the retina of the eye and the blood-forming cells of the bone marrow.

### Payloads

In accordance with various embodiments, any application-appropriate payload may be used as described herein. In accordance with various embodiments, a transgene encodes one or more payloads. As used herein, the terms “payload” and “gene of interest” (GOI) may be used interchangeably. In some embodiments, a payload is or comprises a peptide, a nucleic acid (e.g., shRNA, miRNA, and/or nucleic acid that encodes one or more peptides), and any combination thereof. In some embodiments, integrating gene therapy treatments and/or gene-integrating compositions include a single payload. In some embodiments, integrating gene therapy treatments and/or gene-integrating compositions include two or more payloads (e.g., 3, 4, 5, 6, 7, 8, 9, 10 or more).

For example, in some embodiments, a payload is or comprises a peptide expressed intracellularly or nucleic acid sequence encoding such a peptide (e.g., a transgene). By way of non-limiting example, intracellularly expressed peptides include methylmalonyl-CoA mutase (MUT), phenylalanine hydroxylase (PAH), glucose-6-phosphatase catalytic subunit (G6PC), propionyl-CoA carboxylase, subunit alpha (PCCA), ATP binding cassette subfamily B member 11 (ABCB11), ornithine carbamoyltransferase (OTC), UDP glucuronosyltransferase family 1 member A1 (UGT1A1), Acid alpha-glucosidase (GAA), Lysosomal acid glucosylceramidase (GBA), Frataxin (FTX).

In some embodiments, a payload is or comprises a peptide that is secreted extracellularly and/or a nucleic acid sequence encoding such a peptide (e.g., a transgene). By way of non-limiting example, secreted peptides include human Factor IX (F9), and alpha-1-antitrypsin (SERPINA1).

In some embodiments, a payload is a peptide that has cell-intrinsic or cell-extrinsic activity that promotes a biological process to treat a medical condition.

In some embodiments, a payload may be or comprise a peptide that is normally expressed in one or more healthy tissues, or a nucleic acid sequence encoding such a peptide. For example, in some embodiments, a payload is a peptide that is normally expressed in liver cells. For example, in some embodiments, a payload is a peptide that is normally expressed in muscle cells. For example, in some embodiments, a payload is a peptide that is normally expressed in cells of the central nervous system. For example, in some embodiments, a payload is a peptide that is normally expressed in cells of the eye.

In some embodiments, a payload may be or comprise a peptide that is not normally expressed in one or more healthy tissues (e.g., it is expressed ectopically) or a nucleic acid sequence encoding such a peptide. For example, in some embodiments, a payload is a peptide that is ectopically expressed in liver cells. For example, in some embodiments, a payload is a peptide that is normally expressed in muscle cells. For example, in some embodiments, a payload is a peptide that is normally expressed in cells of the central nervous system. For example, in some embodiments, a payload is a peptide that is normally expressed in cells of the eye.

In some embodiments, a payload my comprise an activation element (e.g., which is activated by an activating agent). In some embodiments, a payload my comprise an deactivation element (e.g., which is activated by an deactivating agent). In some embodiments, an activation or deactivation agent may be or comprise a small molecule,

### Biomarkers

The present disclosure provides integrating gene therapy technologies that generate a detectable biomarker that can act as a proxy for expression of the payload.

In accordance with the present disclosure, expression of an integrated transgene involves production of a transcript that includes at least one translatable open reading frame that encodes a polypeptide separate or separable from payload encoded by the transgene. In some embodiments, translation of the transcript generates a single polypeptide that becomes cleaved to separate the payload from the biomarker; in some embodiments, translation of the transcript generates distinct biomarker and payload polypeptides.

As is described in the present disclosure, use of a variety of biomarkers is contemplated as compatible with various embodiments. In some embodiments, a biomarker is or comprises a detectable moiety that, after translation of a polypeptide encoded by a target site, becomes fused to the polypeptide encoded by the target site. In some embodiments, a biomarker is or comprises a detectable moiety that, after translation of a polypeptide encoded by a target site, becomes fused to the polypeptide encoded by the payload. In some embodiments, associating a biomarker with a payload can be advantageous, for example, when a payload is a modified form of an endogenous protein and therefore would otherwise be difficult or impossible to detect separate and apart from the endogenous version. In some embodiments, a detectable moiety may be or comprise an agent that binds to a biomarker (e.g., an antibody or fragment thereof, for example, an antibody that binds a 2A peptide).

In some embodiments, a biomarker is or comprises a 2A peptide. In some embodiments, a 2A peptide is selected from the group consisting of P2A, T2A, E2A and F2A. In some embodiments, a biomarker may be or comprise a Furin cleavage motif. By way of non-limiting example, an array of Furin cleavage motifs is described in Tian et al., FurinDB: A Databse of 20-Residue Furin Cleavage Site Motifs, Substrates and Their Associated Drugs, (2011), Int. J. Mol. Sci., vol. 12: 1060-1065. By way of specific example, in some embodiments, a 2A peptide may have or comprise the amino acid sequence ATNFSLLKQAGDVEENPGP (SEQ ID NO: 1) and a transgene encoding such a 2A peptide may have or comprise the nucleotide sequence gccaccaacttcagcctgctgaaacaggccggcgacgtggaagagaaccctggcccc (SEQ ID NO: 2). In some embodiments, a 2A peptide will have a sequence that is at least 80% identical to SEQ ID NO:1 or SEQ ID NO:2 (e.g. at least 85%, 90%, 95%, 99% identical). In some embodiments, a 2A peptide or transgene encoding a 2A peptide may be or be generated as described in Kim et al., (2011) High Cleavage Efficiency of a 2A Peptide Derived from Porcine Teschovirus-1 in Human Cell Lines, Zebrafish and Mice, PLoS ONE, vol. 6(4):e18556; Wang et al. (2015) 2A self-cleaving peptide-based multi-gene expression system in the silkworm Bombyx mori, Sci Rep., vol. 5: 16273; Yu et al. (2012) Use of Mutated Self-Cleaving 2A Peptides as a Molecular Rheostat to Direct Simultaneous Formation of Membrane and Secreted Anti-HIV Immunoglobulins. PLoS ONE 7(11): e50438; or Trichas et al. (2008), Use of the viral 2A peptide for bicistronic expression in transgenic mice, BMC Biology, vol. 6:40.

In some embodiments, a biomarker may be or comprise a tag (e.g., an immunological tag), for example, a myc, HA, FLAG, or other tag. In some embodiments, a biomarker may be or comprise an internal ribosome entry site (IRES).

### Detecting Detectable Moiety

As is described herein, detecting (e.g., detecting a signal, such as a biomarker or detectable moiety), as applicable to methods and compositions described herein, may be achieved in any application-appropriate manner. For example, in some embodiments, a step of detecting is or comprises an immunological assay or a nucleic acid amplification assay.

As is described herein, many embodiments include the use of one or more biological samples (e.g., a sample of fluid or tissue taken form a subject), and the manner of detecting a biomarker may vary depending upon the biological sample used in a particular embodiment. In accordance with the present disclosure, any of a variety of biological samples are contemplated as compatible with various embodiments. For example, in some embodiments, a biological sample is or comprises hair, skin, feces, blood, plasma, serum, cerebrospinal fluid, urine, saliva, tears, vitreous humor, or mucus.

In accordance with various embodiments, and depending upon the specific biomarker(s) used, one of skill in the art will envision one or more appropriate methods of detecting or determining the presence and/or quantity/level of a biomarker in a biological sample. In some embodiments, a biomarker may be detected using any of a variety of modalities including fluorescence, radioactivity, chemiluminescence, electrochemiluminescence, colorimetry, FRET, HTRF, isotopic methods, partner binding (e.g., biotin/avidin, antibodies, hybridization), or any other known manner of detecting a biomarker. In some embodiments, a biomarker may be detected through binding of a detectable moiety (e.g., an exogenously added detectable moiety) such as an antibody that includes, for example, a tag in accordance with one or more of the above modalities, or an enzyme (e.g., luciferase, β-gal).

### Applications and Additional Aspects

Those skilled in the art will readily appreciate that the methods described herein can be useful in a variety of applications involving gene therapy. In some embodiments, methods described herein may be useful in assessing whether or not a payload is being over or under expressed, relative to a desired or “normal” level of expression. In some embodiments, methods described herein may be used to predict or characterize a potential adverse reaction to gene therapy (e.g., production of detrimental immune response such anti-drug antibodies and/or cytokine storms), thus potentially allowing for intervention and/or mitigation of the adverse reaction.

It is specifically envisioned that methods and compositions described herein are applicable to any of a variety of diseases, disorders, or conditions. By way of non-limiting examples, some embodiments may be useful in monitoring the course of therapy or other parameter of acidosis/academia (e.g., methylmalonic academia), urea cycle disorder, hemophilia, Crigler-Najjar, acute hepatic porphyria, hereditary ATTR amyloidosis, and/or alpha-1 antitrypsin deficiency (A1ATD), among others.

In accordance with various embodiments, methods and compositions described herein are contemplated as compatible with a variety of gene therapy regimen. For example, in some embodiments, a subject receives a single dose of a gene therapy treatment or gene-integrating composition. In some embodiments, a subject receives multiple doses of one or more gene therapy treatments and/or gene-integrating compositions (e.g., 2, 3, 4, 5, 6, 7, 8, 9, 10 or more).

Additionally, methods and compositions as described herein are contemplated as applicable at any of a variety of times post gene therapy treatment(s) (e.g., hours, days, weeks, or months after the subject receives a gene therapy). Accordingly, in some embodiments, a detecting step is performed 1, 2, 3, 4, 5, 6, 7, 8 or more weeks after the subject has received the gene therapy treatment or gene-integrating composition. In some embodiments, a detecting step is performed at multiple time points after the subject has received the gene therapy treatment or gene-integrating composition. In some embodiments, a detecting step is performed (e.g., multiple times) over a period of at least 3 months after the subject has received the gene therapy treatment or gene-integrating composition.

Surprisingly, it was found that some embodiments are capable of providing benefit (e.g., facilitating monitoring and/or adjustment of therapy) to a subject that is at various stages of life when receiving a gene therapy and, in some embodiments, provided methods may be used as a subject transitions between stages of life. In some embodiments, a subject receives the gene therapy treatment or gene-integrating composition as an infant. In some embodiments, a subject receives the gene therapy treatment or gene-integrating composition before reaching adulthood (e.g., as a child). In some embodiments, a subject receives the gene therapy treatment or gene-integrating composition as an adult.

It is specifically contemplated that methods and compositions as described herein are applicable to a variety of subjects, each potentially having confounding or complicating factors/conditions in addition to those necessitating the application of gene therapy. In addition, some forms of gene therapy are known or suspected of potentially causing problematic reactions (e.g., autoimmune reactions, cytokine storms, etc). Accordingly, in some embodiments, provided methods further comprise monitoring the subject for autoimmune response to the gene therapy. In some embodiments, in some embodiments, provided methods further comprise monitoring the subject for an abnormal cytokine response to the gene therapy (e.g., a cytokine storm).

The present disclosure also encompasses the recognition that gene therapy may need to be adjusted at times (e.g., enhanced or suppressed), and it is contemplated that various embodiments are advantageous in monitoring the need for, and/or successfully making such adjustments. Accordingly, in some embodiments, provided methods further comprise administering an additional treatment (e.g., an activating agent) to the subject if the level of the biomarker is lower than would indicate a therapeutically effective amount of the integrating gene therapy has been achieved. Additionally or alternatively, in some embodiments, provided methods further comprise delivering an additional treatment (e.g., a deactivating agent) to the subject that reduces or inhibits expression of a payload delivered by the gene therapy treatment if the level of the biomarker exceeds a level that is indicative of an optimal or safe level of the payload.

### EXEMPLIFICATION

### Example 1: Exemplary Methods for Biomarker and Payload Detection

The present example demonstrates exemplary methods that can be applied for detection, monitoring and/or analysis of biomarker and/or payload in accordance with the present disclosure. Examples 2-7 employed the methods and materials described herein.

### Genomic DNA Integration (INT) Assay: Long Range qPCR

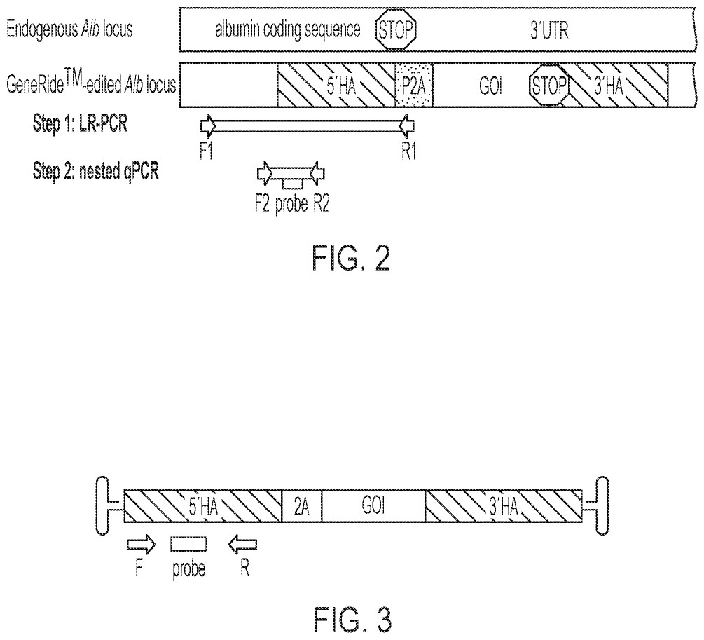

Genomic DNA (gDNA) was isolated from frozen mouse liver tissue with Qiagen's DNeasy kit. Long-range PCR (LR-PCR) product amplified with primers F1/R1 (step 1) was purified with SPRI magnetic beads and used in nested qPCR (step 2) with primer set F2/R2 (FIG. 2). A synthetic dsDNA encompassing a fragment upstream from the 5′ Homology Arm to the gene of interest (GOI) (˜2.1 Kb) was used to generate the standard curve. Standards were run side-by-side with samples. Tfrc was used as a loading control for normalization.

### Episomal DNA Assay

Genomic DNA (gDNA) was isolated from mouse liver with Qiagen's DNeasy kit. Episomal copy numbers were determined by qPCR (FIG. 3) using a standard curve built with linearized episomal plasmid. Tfrc was used as a loading control for normalization. Due to cross-reactivity of the primers with the endogenous Alb gene, the limit of detection of the assay is 2, for the 2 copies of Alb in the genome.

### Fused mRNA Assay

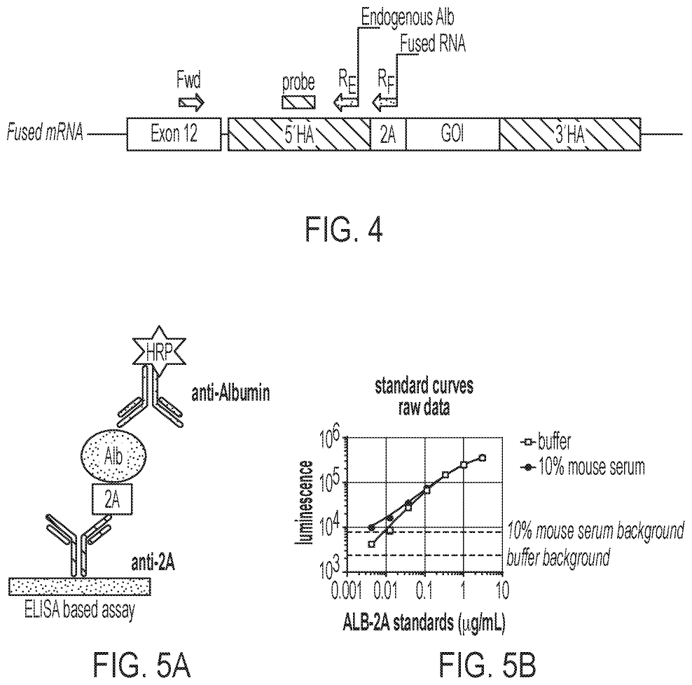

RNA from mouse liver was isolated with Qiagen's RNeasy kit. Fused mRNA copy number was confirmed by ddPCR with primer set Fwd/R_{F} (FIG. 4). Endogenous Alb copy number was measured by ddPCR with primer set Fwd/R_{E} and used for normalization.

### Albumin-2A ELISA

Albumin-2A in plasma was measured by chemoluminescence ELISA, using a proprietary rabbit polyclonal anti-2A antibody for capture and a HRP-labelled polyclonal sheep anti-Albumin antibody (BioRad AHP102P) for detection (FIG. 5A). Recombinant mouse Albumin-2A expressed in mammalian cells and affinity-purified was used to build the standard curve in 10% control mouse plasma to account for matrix effects (FIG. 5B). Casein at 1% (Thermo 37528) was used for blocking and at 0.1% for sample dilution in PBST.

### Albumin ELISA

Total mouse albumin in plasma was measured by chemoluminescence ELISA, using a polyclonal goat anti-mouse albumin antibody (abcam ab19194) for capture and a polyclonal sheep HRP-labelled anti-Albumin antibody (BioRad AHP102P) for detection. Mouse albumin standard was purchased from Sigma (SLBX6058). Casein at 1% (Thermo 37528) was used for blocking and at 0.1% for sample dilution in PBST.

### High-Sensitivity Albumin-2A ELISA

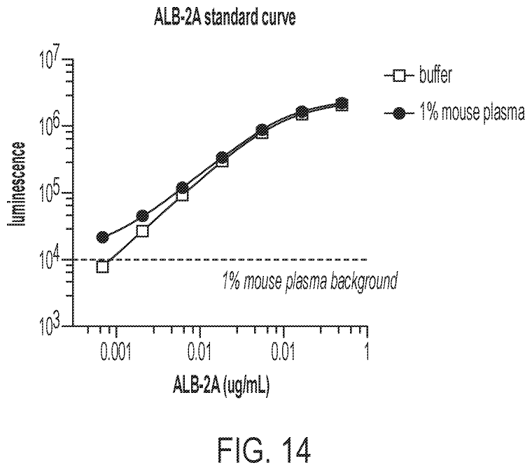

The original Albumin-2A ELISA protocol was optimized to improve the sensitivity of the assay and minimize matrix interference. A proprietary recombinant rabbit monoclonal anti-2A antibody was developed and used for capture, and an HRP-labelled polyclonal goat anti-Albumin antibody (abeam ab19195) used for detection (FIG. 5A). Recombinant mouse Albumin-2A expressed in mammalian cells and affinity-purified with a purity >95% was used as standard to build the calibration curve in ≤1% control mouse plasma or serum (FIG. 14). A buffer consisting of 1% nonfat dry milk was used for blocking, and samples were diluted in 1% BSA in PB ST. The lower limit of detection with this optimized protocol is <1 ng/mL.

### Human Factor IX ELISA

Human Factor IX in plasma was measured by chemoluminescence ELISA, using a monoclonal mouse anti-human Factor IX antibody (Sigma F2645) for capture and a goat polyclonal HRP-labelled anti-human Factor IX antibody (Affinity Biologicals GAFIX-APHRP) for detection. Human Factor IX standard was purchased from abeam (ab62544), and the standard curve was built in 6% control mouse plasma to account for matrix effects. BSA at 3% was used for blocking and at 1% for sample dilution in PBST.

### Cyno A1AT ELISA

Cyno A1AT in plasma was measured by chemoluminescence ELISA, using a goat polyclonal anti-A1AT antibody for capture (MP Biomedical 55030) and a sheep polyclonal HRP-labelled anti-A1AT antibody (abeam ab8768) for detection. Recombinant cyno A1AT expressed in mammalian cells was used to build the standard curve in 10% control mouse plasma to account for matrix effects. BSA at 3% was used for blocking and at 1% for sample dilution in PBST.

### MUT Western Blot

Frozen liver tissues (˜60 mg) were homogenized in lysis buffer (0.5% Igepal-630, 50 mM Tris-HC1 pH 7.5, 150 mM NaCl, supplemented with Roche mini-tablet protease inhibitor cocktail) using MP Biomedicals Lysing Matrix D (#116913050) with two rounds of bead beating (20 sec at 3500 rpm per round). Liver lysates were clarified by centrifugation and total protein was quantified by the BCA assay. Lysates (6 μg/lane) were resolved on a 4-12% NuPAGE BisTris midi-gel (Life Technologies) using MES buffer, before transfer into nitrocellulose membranes on the Trans-Turbo Blot system (Bio-Rad). After blocking in LI-COR Odyssey Blocking Buffer, membranes were incubated with (a) rabbit monoclonal anti-MUT antibody (abcam ab134956) and mouse monoclonal anti-β-actin antibody (abcam ab14128) or (b) rabbit polyclonal anti-2A antibody (proprietary) and mouse polyclonal anti-albumin (abcam ab19194). Following incubation with secondary antibodies (anti-rabbit IRDye800CW and anti-mouse IRDye680CT), blots were scanned in a LI-COR Odyssey system and images were analyzed with ImageStudio software.

### Transgenes and Vectors

Human Factor IX transgene: Codon optimized human F9 cDNA with a P2A coding sequence at its 5′-end, was flanked by homology arms 1.3 Kb upstream and 1.4 Kb downstream to the Alb stop codon (SEQ ID NO: 3).

Human methylmalonyl-CoA mutase transgene: Codon optimized human MUT cDNA with a P2A coding sequence in the 5′ end was flanked by homology arms 1 Kb upstream and 1 Kb downstream to the Alb stop codon (SEQ ID NO: 4).

Mouse methylmalonyl-CoA mutase transgene: Mouse MUT cDNA with a P2A coding sequence in the 5′ end was flanked by homology arms 1 Kb upstream and 1 Kb downstream to the Alb stop codon (SEQ ID NO: 5).

Cynomolgus alpha-1-antitrypsin (cyno-A1AT) transgene: Codon optimized cynomolgus SERPINA1 cDNA with a P2A coding sequence in the 5′end, was flanked by homology arms of 1 Kb (SEQ ID NO: 6) or 750 bp (SEQ ID NO: 7) upstream and downstream to the Alb stop codon.

Vector preparation: All plasmids for rAAV production were prepared using Qiagen's EndoFree Plasmid Gigaprep Kit.

DJ vectors were generated at research-grade scale, using triple transfection in adherent HEK-293 cells with CsC1 gradient purification for hF9 and MUT, and using triple transfection in suspension HEK-293F cells with affinity purification followed by iodixanol gradient for cyno-A1AT. Physical titers were quantified by qPCR.

### In Vivo Studies

All animal procedures were performed in accordance with the Institutional Animal Care and Use Committee (IACUC) guidelines for the care and use of animals in research.

C57BL/6 and FvB/NJ mice were purchased from Jackson Laboratory to serve as breeding pairs to produce offspring for neonatal (p2) and juvenile (p21) injections. 2-day-old mice were injected intravenously (i.v.) via facial vein with ˜10 μL of vector for a final dose of 1e13 or 1e14 vg/kg, or PBS for the vehicle group. 21-day-old mice were injected i.v. via tail vein with ˜100 μL of vector for a final dose of 1e13 or 1e14 vg/kg, or PBS for the vehicle group. Adult mice (>6-week-old) were injected i.v. via tail vein with ˜200 μL of vector for a final dose of 1e13 or 1e14 vg/kg, or PBS for the vehicle group. Animal weight was monitored weekly. In-life blood collections were performed by cheek-bleed, and terminal bleeds by cardiac puncture, except for p7 animals, which were decapitated. At harvest, mice were euthanized by CO₂ inhalation and liver was collected, sectioned and immediately snap frozen.

MUT mouse model (Mut^{-/-}; Tg^{INS-MCK-Mut}) was acquired from Dr. Charles Venditti (National Human Genome Research Institute, National Institutes of Health). Neonatal mice (p1-2) were injected i.v. via facial vein with ˜10 μL of hMUT-DJ vector for a final dose of 1e13, 3e13 or 1e14 vg/kg, or PBS for vehicle.

### Example 2: Detection of Episomal DNA

The present example demonstrates applications of detecting and analyzing episomal DNA in accordance with the methods disclosed herein.

Neonate mice were injected i.v. with a viral vector comprising a P2A coding sequence and a human Factor IX transgene designed to integrate at an endogenous albumin target site (see, human Factor IX transgene described in Example 1).

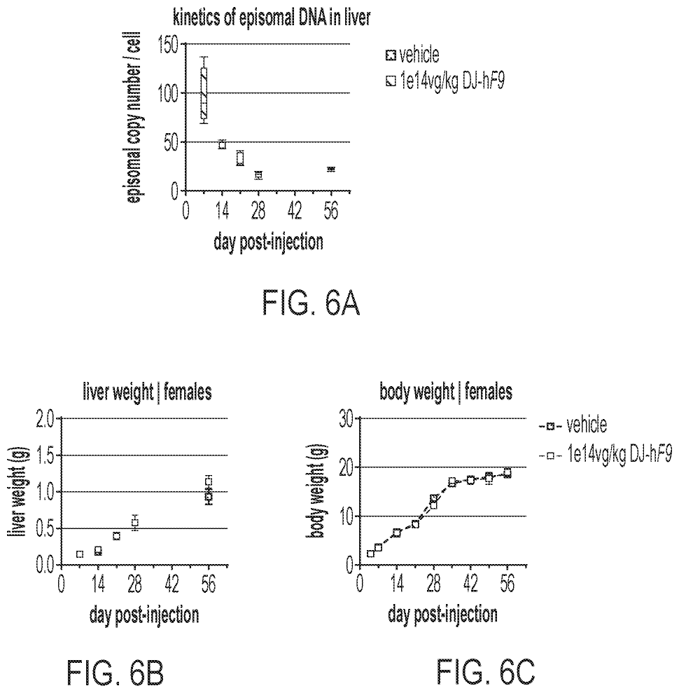

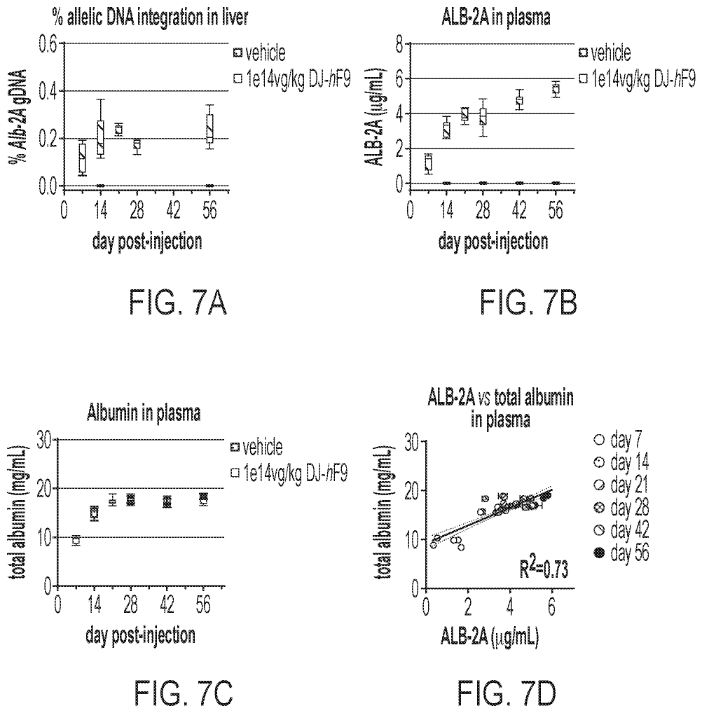

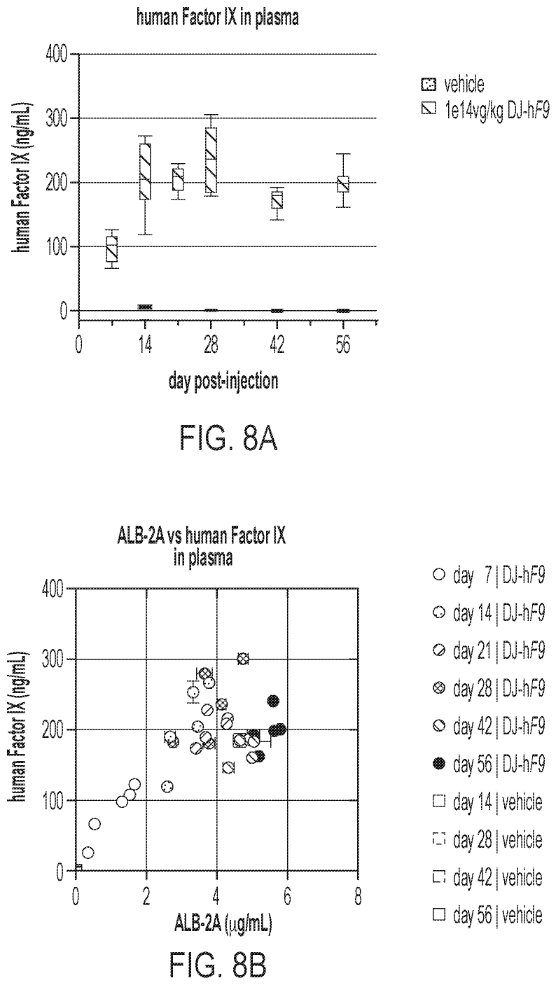

Episomal copy numbers decreased exponentially over time after injection (FIG. 6A). The decrease in episomal copies was observed alongside growth of the liver (from 0.15 g at p7 to 1 g at 8-weeks-old) (FIG. 6B). Despite the decrease in episomal copies per cell over time, transgene expression remained high, which is expected after stable integration of the transgene in the genome within the first week, such as achieved using GeneRide™ (see, e.g., FIG. 7A and FIG. 8A).

These data demonstrate that episomal DNA per cell decreases over time as the liver grows. These data additionally demonstrate that growth and development of the animals is not affected by dosing a high titer of AAV, here 1e14 vg/kg (FIG. 6B-6C). These data also demonstrate that episomal copy numbers of a viral vector delivered in vivo can be a proxy for dosing and tissue growth. Specifically, episomal copy numbers are proportional to the dose to which the animal has been exposed. For example, if an animal is misdosed (which can happen when injecting i.v. neonatal mice), the episomal copies for that animal will be much lower than a fully dosed animal.

### Example 3: Detection of ALB-2A in Plasma and Correlation with Changes in Endogenous Albumin Expression

The present example demonstrates detection and monitoring of 2A-peptide-tagged albumin following in vivo delivery of a nucleic acid element encoding a 2A peptide. The present example also demonstrates that changes in levels of ALB-2A in plasma can be associated with changes in endogenous albumin expression.

Neonate mice were injected i.v. with the viral vector described in Example 2. Genomic integration can be detected as early as after 1 week post-injection, and after 2 weeks it has already reached its plateau (FIG. 7A). ALB-2A in circulation increases over the first 3-4 weeks and then stabilizes (FIG. 7B). The observed increase in plasma ALB-2A is associated with the exponential increase of endogenous albumin after birth (FIG. 7C and FIG. 7D).

These data demonstrate that methods utilized in accordance with the present disclosure allow ready detection and/or analysis of ALB-2A in plasma and that detection and/or analysis of ALB-2A in plasma can be achieved relatively early (e.g., within 1 week) following in vivo delivery of a nucleic acid element encoding a 2A peptide. These data further demonstrate that levels of ALB-2A in plasma can be monitored at multiple time intervals post initial delivery of the nucleic acid element encoding a 2A peptide and that levels of ALB-2A in plasma correlate with changes in levels of an endogenous polypeptide that is encoded at a target site for integration of the 2A peptide (e.g., the albumin target site).

### Example 4: Early Biomarker and Payload Detection and Analysis

The present example demonstrates that methods utilized in accordance with the present disclosure allow early in vivo detection and analysis of a payload following in vivo delivery of a nucleic acid encoding the payload and a nucleic acid encoding a biomarker. The present example also demonstrates that the kinetics of payload expression exhibits similarities to that of biomarker expression.

Neonate mice were injected i.v. with the viral vector described in Example 2. Analysis of plasma levels of human Factor IX, which is expressed from the integrated transgene, reveals a similar early kinetics to ALB-2A (FIG. 8A-8B, respectively). Similar to the kinetics of ALB-2A in plasma, the levels of Factor IX increased as endogenous albumin increased after birth.

These data demonstrate that expression of a payload (e.g., Factor IX) can be detected and/or analyzed relatively early (e.g., within 1 week) following delivery via a viral vector. These data also demonstrate similar expression kinetics of a payload with that of plasma levels of a biomarker delivered with the payload (e.g., 2A peptide) at multiple time intervals following the initial delivery of the payload.

### Example 5: Integration Efficiency, Plasma ALB-2A and Transgene are not Affected by the Age of Animals at Dosing

The present example demonstrates that methods utilized in accordance with the present disclosure can be applied to gene therapy treatments administered at different ages of subjects receiving such treatments, including at very young (e.g., infant) and pre-adult stages of development. The present example further demonstrates that the methods described herein can be applied for analysis of gene therapy that is delivered to tissues with either low or high growth.

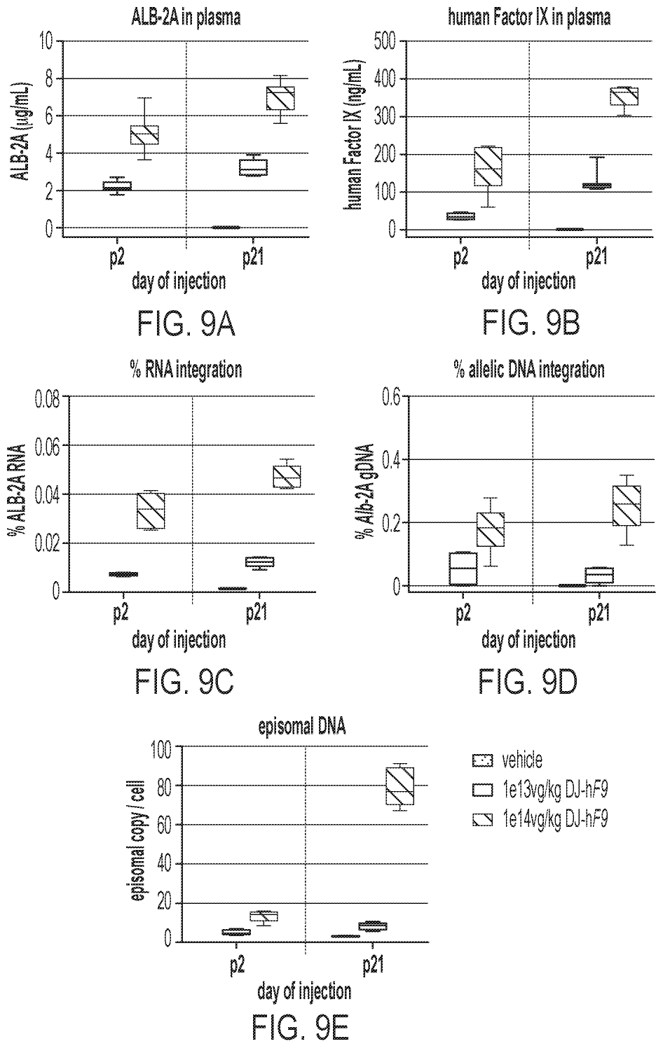

Neonate (p2) or juvenile (p21) mice were injected i.v. with the viral vector described in Example 2, and harvested 8 weeks post-injection. Regardless of the age at dosing tested, ALB-2A and human Factor IX in plasma (FIG. 9A and 9B, respectively) as well as RNA integration of the 2A biomarker (FIG. 9C) are readily detectible. Integration efficiency in genomic DNA is not significantly affected by the age at which the animals are dosed (FIG. 9D). Episomal copy numbers after 8-weeks post-injection are still high in animals dosed at p21 (FIG. 9E), as would correspond to the lower growth of the liver in juvenile animals.

These data demonstrate that biomarker (e.g. 2A) and payload (e.g., Factor IX) expression can be detected and/or analyzed following delivery of said biomarker and payload to a subject at different stages of development, including at different ages of the subject and/or different stages of growth for a tissue in tissue-directed delivery. In the present example, use of a DJ vector targeted nucleic acid delivery of the 2A and payload to the liver.

### Example 6: Biomarker Levels as a Proxy for Payload Levels at Different Ages of Administration

The present example demonstrates that methods utilized in accordance with the present disclosure enable detection and analysis of a biomarker as a proxy for levels of payload. The present example also demonstrates that use of biomarker as a proxy can be practiced for delivery of payload and biomarker at diverse age groups of subjects.

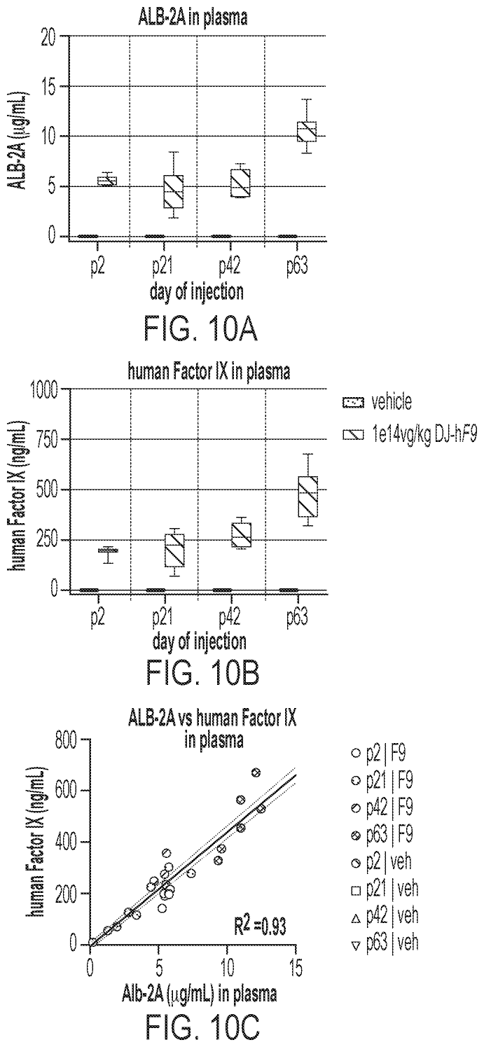

Neonate (p2), juvenile (p21) or adult (p42 and p63) mice were dosed i.v. with the viral vector described in Example 2 and harvested 4 weeks post-injection. Ready detection of ALB-2A in plasma (FIG. 10A) and human Factor IX (FIG. 10B) was observed at each age group tested relative to vehicle treatment. Relative levels of ALB-2A were indicative of human Factor IX levels among the tested age groups (FIG. 10C).

These data demonstrate that detection of a biomarker (e.g. ALB-2A) is indicative of expression of a payload (e.g., Factor IX). These data further illustrate detection and analysis of a biomarker can be useful as a proxy for levels of payload delivered to a subject. Moreover, such measure of biomarker levels as a proxy for payload levels can be useful for analysis of payload delivered to subjects at diverse age groups.

### Example 7: Biomarker Levels as a Proxy for Payload Levels Is Observed with Changes in Vector Design

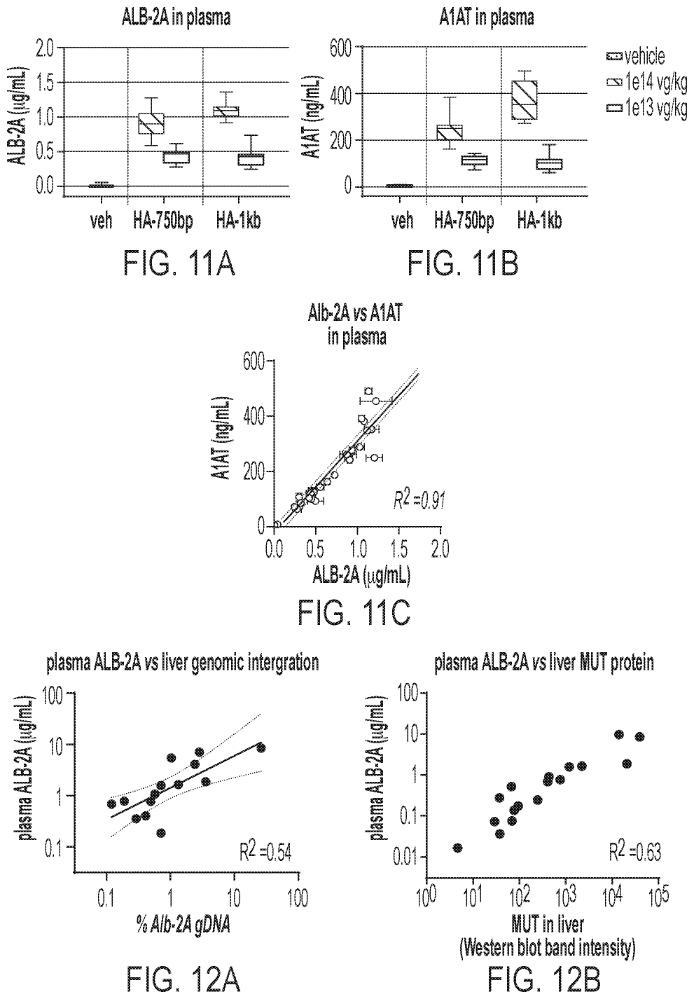

Neonate mice were injected i.v. with a viral vector comprising a P2A coding sequence, a cynomolgus SERPINA1 cDNA, and homology arms of 1 Kb or 750 bp, designed to integrate at an endogenous albumin target site (see, cynomolgus alpha-1-antitrypsin (cyno-A1AT) transgene described in Example 1). Animals were harvested 6 weeks post-injection. Plasma levels of ALB-2A (FIG. 11A) were detected after delivery with viral vectors comprising homology arms of both 750 bp and 1 Kb. Plasma levels of cynomolgus A1AT, which is expressed from the integrated transgene, were also readily detectable for both sets of homology arms tested (FIG. 11B). Relative levels of ALB-2A were indicative of cynomolgus A1AT levels (FIG. 11C).

### Example 8: Plasma Biomarker Levels as a Proxy for Integration and Expression of Cell-Intrinsic Payload

A murine model of MMA called Mut^{-/-}; Tg^{INS-MCK-Mut} mice (referred to herein as MCK-Mut) was used in the present example. In this mouse model, a functional copy of the mouse Mut gene is under the control of the creatine kinase promoter, enabling Mut expression in muscle cells. Neonatal MCK-Mut mice (p2) were injected i.v. with different doses of a viral vector comprising a P2A coding sequence and a codon optimized human MUT cDNA (see, Human methylmalonyl-CoA mutase transgene described in Example 1). Animals were harvested over a period of 3 months. Plasma ALB-2A and genomic integration in liver were readily detectable (FIG. 12A). Relative levels of ALB-2A in the liver and in plasma were indicative of MUT protein levels in the liver (FIG. 12B).

These data demonstrate that methods utilized in accordance with the present disclosure allow expression of a cell-intrinsic payload (e.g., MUT), which expression can be evaluated by detection and analysis of expression of a detectable moiety fused to a polypeptide encoded by a target site gene (e.g., ALB-2A). These data further demonstrate that changes in expression of a cell-intrinsic payload (e.g., MUT) can be reflected in analogous changes in plasma levels of a detectable moiety (e.g., 2A peptide), such that detection of plasma levels of a detectable moiety can act as a proxy for detection of a cell-intrinsic payload. Additionally, these data demonstrate the ability to monitor levels of a cell-intrinsic payload in real-time, without requiring detection of the cell-intrinsic payload itself (e.g. via liver biopsy).

### Example 9: Biomarker Levels as a Proxy for Changing Levels of Payload over Time

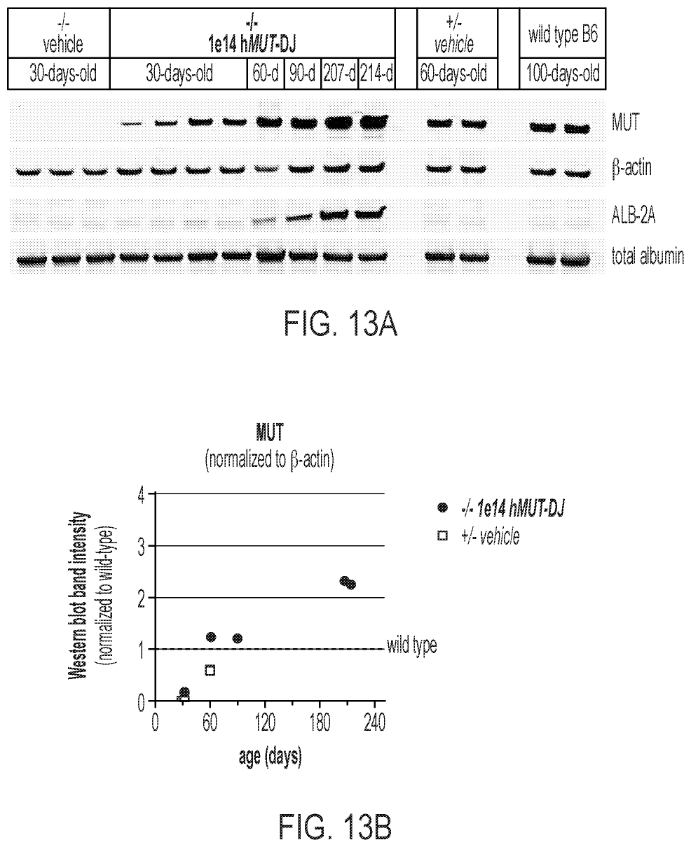

Neonatal MUT-MCK mice (p2) were injected i.v. with 1e14 vg/kg of DJ-hMUT, and harvested over a period of 7 months. Hepatocytes edited by GeneRide express functional MUT, which gives them selective growth advantage over Mut^{-/-} endogenous hepatocytes. As a result, the gene-edited population is expected and observed to grow faster. This expansion can be detected by the increased levels of the transgene as well as the ALB-2A (FIG. 13A-FIG. 13B).

These data illustrate that levels of a payload can increase over time after a single dose delivery in tissue lacking wild-type expression of a protein corresponding to the polypeptide encoded by the payload. These data further demonstrate that increased levels of a biomarker track with the increased levels of the payload. One of skill in the art would be able to apply the methods disclosed herein to detect and analyze a plasma biomarker, such as ALB-2A, as a proxy for the expression of an intracellular payload (e.g., MUT).

### Example 10: Long-Term Biomarker and Payload Detection and Analysis

The present example demonstrates that methods utilized in accordance with the present disclosure allow extended in vivo assessment of expression of a payload following in vivo delivery of a nucleic acid encoding the payload and a nucleic acid encoding a biomarker. The present example also demonstrates validation of an assay in that the kinetics of payload expression exhibits similarities to that of biomarker expression.

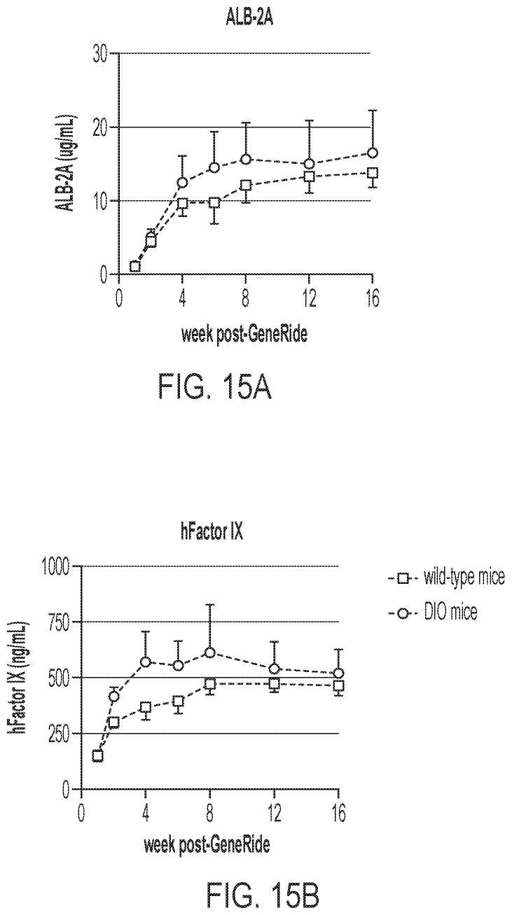

Adult mice were injected i.v. with the viral vector described in Example 2. Analysis of plasma levels of human Factor IX, which is expressed from the integrated transgene, reveals similar kinetics to ALB-2A from week 1 to week 16 (FIG. 15A-15B), rapidly increasing within the first weeks after injection until reaching a steady state.

These data demonstrate that expression of a payload (e.g., Factor IX) can be assessed over an extended period of time (e.g., up to 16 weeks) following delivery via a viral vector. Importantly, these data validate similar expression kinetics of a payload and that of plasma levels of a biomarker delivered with the payload (e.g., 2A peptide) at multiple time intervals following the initial delivery of the payload.

### Example 11: Dose-Dependent Biomarker, gDNA, and Payload Detection and Analysis

The present example demonstrates that methods utilized in accordance with the present disclosure allow dose-dependent detection and analysis of a payload following in vivo delivery of a nucleic acid encoding the payload and a nucleic acid encoding a biomarker.

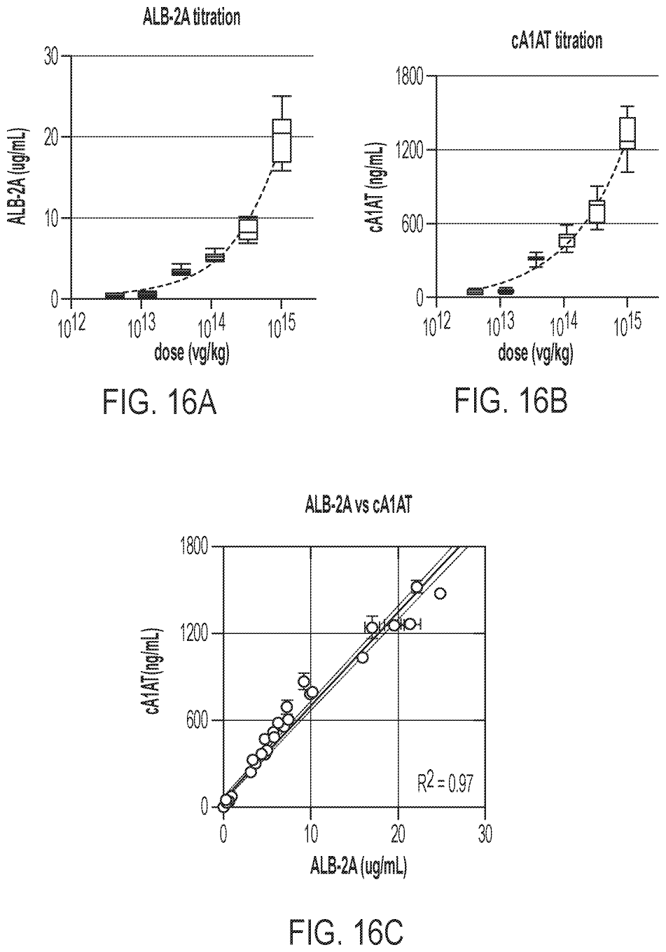

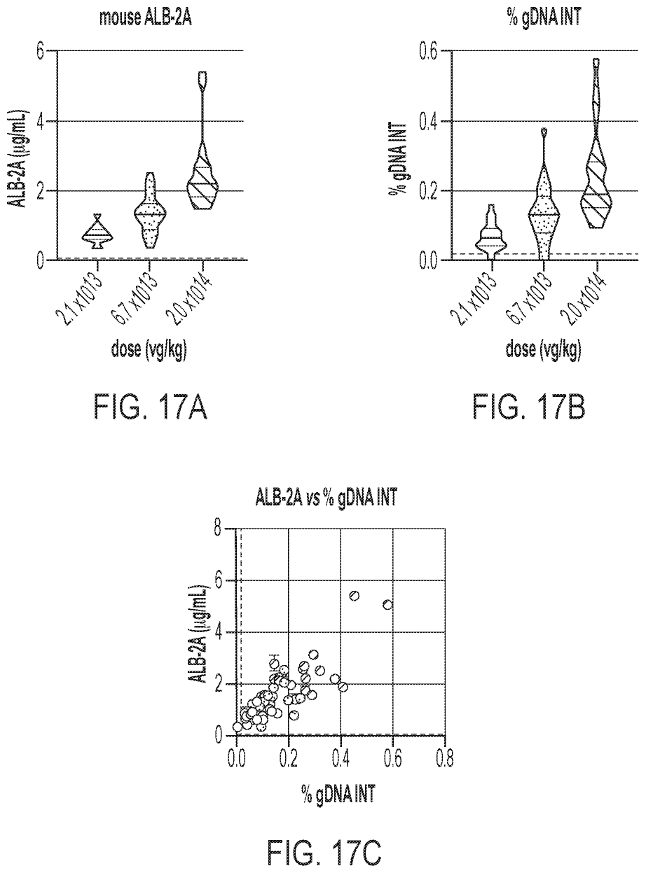

These data demonstrate that methods utilized in accordance with the present disclosure allow dose-dependent expression of a payload, here a cell-intrinsic payload (e.g., mMUT) or a secreted payload (e.g., cA1AT), which expression can be evaluated by detection and analysis of expression of a detectable moiety fused to a polypeptide encoded by a target site gene (e.g., ALB-2A). These data further demonstrate that dose-dependent changes in expression of a secreted payload (e.g., cA1AT) can be reflected in analogous changes in plasma levels of a detectable moiety (e.g., 2A peptide), such that detection of plasma levels of a detectable moiety can act as a proxy for detection of a secreted payload (FIG. 16A-C). Additionally, these data demonstrate that genomic integration levels for a cell-intrinsic payload can also correlate with analogous changes in plasma levels of a detectable moiety (FIG. 17A-C).

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of monitoring gene therapy, the method comprising a step of:
detecting, in a biological sample from a subject who has received integrating gene therapy treatment, a level or activity of a biomarker generated by integration of the integrating gene therapy treatment, as a surrogate for one or more characteristics of the status of the gene therapy treatment, wherein the one or more characteristics of the status of the gene therapy treatment is selected from the group consisting of level of a payload, activity of a payload, level of integration of the gene therapy treatment in a population of cells, and combinations thereof

2. A method of monitoring delivery, level and/or activity of a payload in a subject who has received a gene-integrating composition that delivers the payload, the method comprising a step of:
detecting, in a biological sample from the subject, level or activity of a biomarker generated by integration of the gene-integrating composition, as a surrogate for delivery, level and/or activity of the payload.

3. The method of claim 1 or claim 2, wherein the payload is or comprises a peptide expressed intracellularly.

4. The method of claim 1 or claim 2, wherein the payload is or comprises a peptide that is secreted extracellularly.

5. The method of any one of claims 1-4, wherein the payload is or comprises a peptide that has cell-intrinsic or cell-extrinsic activity that promotes a biological process to treat a medical condition.

6. The method of any one of claims 1-5, wherein the payload is or comprises a peptide that is normally expressed in liver cells.

7. The method of any one of claims 1-5, wherein the payload is or comprises a peptide that is ectopically expressed in liver cells.

8. The method of any one of claims 1-5, wherein the payload is or comprises methylmalonyl-CoA mutase or human Factor IX.

9. The method of any one of the above claims, wherein the integrating gene therapy treatment or gene-integrating composition achieves integration of a nucleic acid element comprising a sequence that encodes the payload into a target site in the genome of the subject.

10. The method of claim 9, wherein the target site encodes a polypeptide.

11. The method of claim 10, wherein integration of the nucleic acid element occurs at the 5′ or 3′ end of a gene that encodes the polypeptide.

12. The method of claim 9, wherein the target site encodes albumin.

13. The method of any one of claims 9-11, wherein integration of the nucleic acid element does not significantly disrupt expression of the polypeptide encoded at the target site.

14. The method of any one of the above claims wherein the biological sample is or comprises hair, skin, feces, blood, plasma, serum, cerebrospinal fluid, urine, saliva, tears, vitreous humor, or mucus.

15. The method of any one of the above claims wherein the step of detecting comprises an immunological assay or a nucleic acid amplification assay.

16. The method of any one of the above claims wherein the biomarker comprises a detectable moiety that, after translation of the polypeptide encoded by the target site, becomes fused to the polypeptide encoded by the target site.

17. The method of any one of the above claims wherein the biomarker comprises a detectable moiety that, after translation of the polypeptide encoded by the target site, becomes fused to the polypeptide encoded by the payload.

18. The method of any one of the above claims wherein the biomarker comprises a detectable moiety that is a 2A peptide or a Furin cleavage motif.

19. The method of claim 18, wherein the 2A peptide is selected from the group consisting of P2A, T2A, E2A and F2A.

20. The method of any one of the above claims wherein the subject receives a single dose of the gene therapy treatment or gene-integrating composition.

21. The method of any one of the above claims wherein the detecting step is performed 1, 2, 3, 4, 5, 6, 7, 8 or more weeks after the subject has received the gene therapy treatment or gene-integrating composition.

22. The method of any one of the above claims wherein the detecting step is performed at multiple time points after the subject has received the gene therapy treatment or gene-integrating composition.

23. The method of any one of the above claims wherein the detecting step is performed over a period of at least 3 months after the subject has received the gene therapy treatment or gene-integrating composition.

24. The method of any one of the above claims wherein the subject receives the gene therapy treatment or gene-integrating composition as an infant.

25. The method of any one of claims 1-23, wherein the subject receives the gene therapy treatment or gene-integrating composition before reaching adulthood.

26. The method of any one of claims 1-23 wherein the subject receives the gene therapy treatment or gene-integrating composition as an adult.

27. The method of any one of the above claims wherein the method further comprises monitoring the subject for autoimmune response to the gene therapy.

28. A method of determining one or more characteristics of the status of gene therapy treatment in a subject who has received an integrating gene therapy treatment, said method comprising:
a) providing a biological sample from the subject;
b) determining a level of a biomarker, wherein the biomarker is generated by integration of the gene therapy in the genome of the subject; and
c) based on the determined level of the biomarker, establishing one or more characteristics of the status of gene therapy treatment in the subject, wherein the determined level of the biomarker corresponds one or more characteristics of the status of gene therapy treatment.

29. A method of delivering a gene therapy treatment to a subject in need thereof, comprising the steps of:
a. administering an integrating gene therapy treatment to the subject; and
b. determining in a biological sample from the subject a level of a biomarker that is generated by integration of the gene therapy treatment in the genome of the subject.

30. The method of claim 29, further comprising
administering an additional treatment to the subject if the level of the biomarker is lower than would indicate a therapeutically effective amount of the integrating gene therapy has been achieved.

31. The method of any one of claims 28-30, wherein the integrating gene therapy treatment achieves integration of a nucleic acid element comprising a sequence that encodes a payload into a target site in the genome of the subject.

32. The method of claim 31, wherein the target site encodes a polypeptide.

33. The method of claim 32, wherein integration of the nucleic acid element occurs at the 5′ or 3′ end of a gene that encodes the polypeptide.

34. The method of claim 31, wherein the target site encodes albumin.

35. The method of any one of claims 31-33, wherein integration of the nucleic acid element does not significantly disrupt expression of the polypeptide encoded at the target site.

36. The method of any one of claims 28-35 wherein the biological sample is or comprises hair, skin, feces, blood, plasma, serum, cerebrospinal fluid, urine, saliva, tears, vitreous humor, or mucus.

37. The method of any one of claims 28-36, wherein the step of determining comprises an immunological assay or a nucleic acid amplification assay.

38. The method of any one of claims 28-37, wherein the biomarker comprises a detectable moiety that, after translation of the polypeptide encoded by the target site, becomes fused to the polypeptide encoded by the target site.

39. The method of any one of claims 28-38, wherein the biomarker comprises a detectable moiety that, after translation of the polypeptide encoded by the target site, becomes fused to the polypeptide encoded by the payload.

40. The method of any one of claims 28-39, wherein the biomarker comprises a detectable moiety that is a 2A peptide.

41. The method of claim 40, wherein the 2A peptide is selected from the group consisting of P2A, T2A, E2A and F2A.

42. The method of any one of claims 28-41, wherein the subject receives a single dose of the gene therapy treatment.

43. The method of any one of claims 28-42, wherein the determining step is performed 1, 2, 3, 4, 5, 6, 7, 8 or more weeks after the subject has received the gene therapy treatment.

44. The method of any one of claims 28-43, wherein the determining step is performed at multiple time points after the subject has received the gene therapy treatment.

45. The method of any one of claims 28-44, wherein the determining step is performed over a period of at least 3 months after the subject has received the gene therapy treatment.

46. The method of any one of claims 28-45, wherein the subject receives the gene therapy treatment as an infant.

47. The method of any one of claims 28-45, wherein the subject receives the gene therapy treatment before reaching adulthood.

48. The method of any one of claims 28-45, wherein the subject receives the gene therapy treatment as an adult.

49. The method of any one of claims 28-48, wherein the method further comprises monitoring the subject for autoimmune response to the gene therapy.

50. The method of any one of claims 1-49, wherein the method further comprises delivering an additional treatment to the subject that reduces or inhibits expression of a payload delivered by the gene therapy treatment if the level of the biomarker exceeds a level that is indicative of an optimal or safe level of the payload.


SEQUENCE LISTING (7 sequences)

SEQ ID NO: 1 - protein, 19 aa, Artificial Sequence
  synthetic polypeptide
ATNFSLLKQAGDVEENPGP

SEQ ID NO: 2 - DNA, 57 bp, Artificial Sequence
  Synthetic polynucleotide
gccaccaacttcagcctgctgaaacaggccggcgacgtggaagagaaccctggcccc

SEQ ID NO: 3 - DNA, 4172 bp, Artificial Sequence
  Synthetic polynucleotide
aggttcgaaccctgctgaagggagaggttccaatactacaaaatgtagcgggatattgtcatcacctttggggacatgtcatcatggtccccagacagagttacaaaactcatcccctacacagcactatgtctctggtactgtttgttctacagatgtcaacaacagaggcccagccatctcctattgcttggcttgtcagtctttctagcctccccattattaatttcaaatggggcaggtgttaggagggcaaaaatccacatattaagtgcaaagcctttcaggagatttcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggccctgctagccagcgcgtgaacatgattatggccgagagccctggcctgatcaccatctgcctgctgggctacctgctgagcgccgagtgtaccgtgttcctggaccacgagaacgccaacaagatcctgaacagacccaagagatacaacagcggcaagctggaagagttcgtgcagggcaacctggaacgcgagtgcatggaagagaagtgcagcttcgaagaggccagagaggtgttcgagaacaccgagagaaccaccgagttctggaagcagtacgtggacggcgaccagtgcgagagcaacccttgtctgaacggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgctgtgcctttcccatgcggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgctaagcctggccagttcccgtggcaggtggtgctgaacggaaaggtggacgccttctgcggcggctccatcgtgaacgagaagtggatcgtgacagccgcccactgcgtggaaaccggcgtgaagatcacagtggtggccggcgagcacaacatcgaggaaaccgagcacacagagcagaaaagaaacgtgatcaggatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggacagagccacctgtctgagaagcaccaagttcaccatctacaacaacatgttctgcgctggcttccacgagggcggcagagactcttgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagctgacataagctagcttagcctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacat

SEQ ID NO: 4 - DNA, 4312 bp, Artificial Sequence
  Synthetic polynucleotide
actccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccccctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgtt

SEQ ID NO: 5 - DNA, 4307 bp, Artificial Sequence
  Synthetic polynucleotide
ttacttggtgggcagatgacacactgatagatgttgtgacaccctctcaccccaactcgtgcatgtgtgatataagttgtgccctcttcatcactggtccctcagagtccccaagtgtcaaagtcaggggtctcaccatttctgaggacctacatattacctatgattacaaatgaaagctgtcttctcagaatgctacaaaactttcaattctttctaaaactgaaataattttgaaatagccttatgtctataggttctttgcttgaataaaaaaataagttttttgttttcatatggagaaacaatttttttgccaaagccttccattattgccctattttaaaaaattctcttctgcaagattataaacaaaattgatcaacatagctgtgtgcagttggctatattttcaaaggaggctgctttgtcataagaagataaattctgttaaagcttagcatggggcacatcagaaatttggaagtggatcacttattatgaaacagaacccgttgtatcttctgttgaaatggattcacagaatgattgggtcaaaccacatggaatcattgatacacaactgttttgacccaccccccaaatattccttcatatggtttcaacttcatccagaggataagcttgactaggctaacatggaggactccacatagtggttcatgtaagtcagagcaaaaccagggctaactcctttgctctggtttgaatttttaattttggttccttctctccctggtttggtctccttttgtgtaaattgcattaacctagacctttatatgttcagtttcttatctacaaatggaacccaaactgatgaccatacgtgaagacctaagagtgccagctggtgcttgtctgtatggctcttgtcaaatggttatcagtcttgatcgttttaggaaaaatgattatgattatgttttttaagtcccaagtatagttacctgagaaggttgtggttgtgatgtgtttacacagactgctgcttctctgccaaacacttctcaatatcatcaagcacttggacagcagctcttggaatccgggttccaggaccaaagacgttggaaacaccaacttcatacagaaattcataatcctgtggtggaatcacgcccccacacatgacaaggatatctggccgccccagggcggtgagttctttgataagctcaggaacgagggttttatgaccagcagcaagtgtgctgacacccacagcatggacatctgcatccacagcttgctgcgccacttcacggggagtctgaaaaagagggcctatgtccacatcaaaaccaagatcagcaaatcctgtagcaatgaccttggctcccctgtcatggccatcttgtcccatttttgccacaagaagacgaggtctgcgaccttcacgttccatgaatttattaactctcttgatggcagatgtgatctctttactttctccaaactcctgccgatatgctccactcaccatacgatcattagctttatgctcaccaaatacttttttcaaggcatccgtgatttctccaactgtacatcttgcacgagctgcatccactgccagagccagaatattgccatctccactggcagcacactgggtaagtgcactgagacactgctcagccaaagcttgatccctgctggatttaatcttcttgagtttttcaatctgcttcttacgcactgaagtgttgtcaatggccaggacctccacagagtcttctttttccaactgatacttatttactccaacaattacctcagaaccagaatctattctagcttgtcttcgggcagcacattcttcaatgcgaagtttagggattccttcagctacagctttggccattccacccatttcttcaacttcatatatcaacttcagagcagcctcataaacgtcatttgtgagcgactccatcatgtacgaccctccccaaggatccgccactttggggatcccagattcttcctgaatgatgatctgtgtgttccgagcaatccgggcacttttcacagtgggcaaacccagcgcttcatcaaaagagttcgtatgcaaagactgggttcctccaaacacagctgccatggcttcgattgcagtgcggacaatgttattgtaaggatcctgctcagtaagtgaccaccccgatgtctggcagtgtgctcttagaagaagagatttagagtttttaggctggaacattttttctattaagtgagcccacagtcttctcccagctcgcatcttggctatttccatgtagaagttcattccaattccccagaagaaagacaaccttggtgcaaattcatcaattgtgagtccagcctggagtccagttctgcagtactccaacccatctgcgatggtataggccagttctaaaatggcatcagctcctgcttcctgcatatggtacccgctaatcgaaatggaattaaattttggcatgtgctgtgctgtgtattggaaaatgtcagcaataattttcatcgatggctctgggggaaaaatataagtatttctgaccataaactcctttaggatgtcattctgaattgtgccagtgagcttctccttcggcacaccttgctcttccccagttactataaatgttgccaggactgggatgacagctccgttcatagtcatggaaacggacattttttctaaaggaatgccatcaaataggattttggtgtcttctacagtgtcaatagcaactccagccattccaacatctccacgaactctggggttgtctgaatcataaccacgatgtgttgccaagtcaaaggcaacagacaacccctgctgaccagccttaatattgtccttatagaatttattgctttcttccacagtactaaagcctgcatactgacggatggtccagggcctataggtatacatggtaggatatggtccccgtgtgaatggcttcactcctggaagttcttcgggtaagtccagagtatctgccctggaatataagggctttatagagatcccttctggggtgtgccatataaggtcctctgggtttttgcctttcagctgctttttggccagtacagcccattctgggtgaaggggttgctgctggtgtagaaggcgtttccatctggaagctgatggaatgtttagctgcttcaggtaatggggcgatagcaaaaaaagttgattcttagctctcaaggggccagggttctcttccacgtcgccggcctgtttcagcaggctgaagttggtggcgccgctgccggctaaggcgtctttgcatctagtgacaaggtttggaccctgaaaacaaagaaatgatggatgttagaaaagtattagcaggactgttaggatttgcacttcatagccataggcaatgacatagtaaacgtacgtgccttgcattgcttaattacagttctacttagaattcctaaagcaggctctctgacatctgttgtgcccaaaataagaagataaactgttgttaggccatgggagggaatgtgaaatgattcagatatataatggtaactgtccattcatgtgtaaaataactcaattcaaaaagaaacatttcagggcaaggtttaaatgagttcatgattctaggctttgcaagtatttctaactatgctcttctgtgtcacaaatttgaaaccaaatagtgataataggtatttaataggcagcagtgtggcagaaaataaaaattaaaagctacgtaagtcagtttctattacagttacgtagagaacaacatctagattggtggagttattcagtgtgtgagttccagagagctacacctgaccacgtgcacagaaaatggaaactgaacatcgtcaaaatgcaaaaacgtttctgacctcagtcgagaagcaggtgtccttgtcagcagccttgcaacatgtatccaggaactgtgcaaagtcatccatgacagtcttcagttgctccgctgtagccttgggcttgtgcttcaccagctcagcaagagccctgcagtaggacagaaagcaagaagagcaaggagacatttaccccagtgcaggaaagtaagataggaaggaatgtgaggccacagtttaaggctataaatcttggagccaagtaactttggctttgaggattaagcatgccactgccaacttaggaatcttggctgtgtaggaggggagtagcagttgtcactttgctaggactagctagatcaaatagaataatcgatgccagtcacacgggttttgtctagtttcag

SEQ ID NO: 6 - DNA, 3344 bp, Artificial Sequence
  Synthetic polynucleotide
ctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggcagcggcgccaccaacttcagcctgctgaaacaggccggcgacgtggaagagaaccctggcccctcttctgtctcatggggcgtcctcctgctggctggcctgtgctgcctgctccccggctctctggctgaggatccccagggagatgctgcccaaaaaacggatacatccctccatgatcaagaccacccaaccctcaacaagatcacccccagcctggctgagttcggcttcagcctataccgccagctggcacaccagtccaacagcaccaatatcttcttctccccagtgagcatcgctacagcctttgcaatgctctccctggggaccaaggctgacactcacagtgaaatcctggagggcctgaatttcaacgtcacggagattccggaggctcaggtccatgaaggcttccaggaactcctccataccctcaacaagccagacagccagctccagctgaccaccggcaacggcctgttcctcaacaagtcactcaaagtagtggataagtttttggaggatgtcaaaaaactgtaccactcagaagccttctctgtcaactttgaggacaccgaagaggccaagaaacagatcaacaattacgtggagaaggaaactcaagggaaaattgtggatttggtcaaggagcttgacagagacacagtttttgctctggtgaattacatcttctttaaaggcaaatgggagagaccctttgacgttgaggccaccaaggaagaggacttccacgtggaccaggcgaccaccgtgaaggtgcccatgatgaggcgtttaggcatgtttaacatctaccactgtgagaagctgtccagctgggtgctgctgatgaaatacctgggcaatgccaccgccatcttcttcctgcctgatgaggggaaactgcagcacctggaaaatgaactcacccatgatatcatcaccaagttcctggaaaatgaaaacagcaggtctgccaacttacatttacccagactggccattactggaacctatgatctgaagacagtcctgggccacctgggtatcactaaggtcttcagcaatggggctgacctctcagggatcacggaggaggcacccctgaagctctccaaggccgtgcataaggctgtgctgaccatcgatgagaaagggactgaagctgctggggccatgtttttagaggccatacccatgtctattccccccgaggtcaagttcaacaaaccctttgtcttcttaatgattgaacaaaataccaagtctcccctcttcatgggaaaagtggtgaatcccacccagaaagagcagaagctgatcagcgaggaggacctgtaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaa

SEQ ID NO: 7 - DNA, 2844 bp, Artificial Sequence
  Synthetic polynucleotide
ctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggcagcggcgccaccaacttcagcctgctgaaacaggccggcgacgtggaagagaaccctggcccctcttctgtctcatggggcgtcctcctgctggctggcctgtgctgcctgctccccggctctctggctgaggatccccagggagatgctgcccaaaaaacggatacatccctccatgatcaagaccacccaaccctcaacaagatcacccccagcctggctgagttcggcttcagcctataccgccagctggcacaccagtccaacagcaccaatatcttcttctccccagtgagcatcgctacagcctttgcaatgctctccctggggaccaaggctgacactcacagtgaaatcctggagggcctgaatttcaacgtcacggagattccggaggctcaggtccatgaaggcttccaggaactcctccataccctcaacaagccagacagccagctccagctgaccaccggcaacggcctgttcctcaacaagtcactcaaagtagtggataagtttttggaggatgtcaaaaaactgtaccactcagaagccttctctgtcaactttgaggacaccgaagaggccaagaaacagatcaacaattacgtggagaaggaaactcaagggaaaattgtggatttggtcaaggagcttgacagagacacagtttttgctctggtgaattacatcttctttaaaggcaaatgggagagaccctttgacgttgaggccaccaaggaagaggacttccacgtggaccaggcgaccaccgtgaaggtgcccatgatgaggcgtttaggcatgtttaacatctaccactgtgagaagctgtccagctgggtgctgctgatgaaatacctgggcaatgccaccgccatcttcttcctgcctgatgaggggaaactgcagcacctggaaaatgaactcacccatgatatcatcaccaagttcctggaaaatgaaaacagcaggtctgccaacttacatttacccagactggccattactggaacctatgatctgaagacagtcctgggccacctgggtatcactaaggtcttcagcaatggggctgacctctcagggatcacggaggaggcacccctgaagctctccaaggccgtgcataaggctgtgctgaccatcgatgagaaagggactgaagctgctggggccatgtttttagaggccatacccatgtctattccccccgaggtcaagttcaacaaaccctttgtcttcttaatgattgaacaaaataccaagtctcccctcttcatgggaaaagtggtgaatcccacccagaaagagcagaagctgatcagcgaggaggacctgtaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatag